(19) United States
(12) Patent Application Publication
(10) Pub. No.: US 2023/0346727 A1
(43) Pub. Date: Nov. 2, 2023

(54) COMPOUNDS FOR THE TREATMENT OF HEMOPHILIA

(71) Applicants: UNIVERSITE GRENOBLE ALPES, Saint Martin d'Heres (FR); CENTRE NATIONAL DE LA RECHERCHE SCIENTIFIQUE, Paris (FR); CENTRE HOSPITALIER UNIVERSITAIRE GRENOBLE ALPES, La Tronche (FR); INSTITUT POLYTECHNIQUE DE GRENOBLE, Grenoble (FR)

(72) Inventors: Aline THOMAS, Meylan (FR); Marie-Claire DAGHER, Fontaine (FR); Muriel JOURDAN, Saint Martin d'Heres (FR); Romain NAVARRO, Grenoble (FR); Benoît POLACK, Saint Martin le Vinoux (FR); Raphaël MARLU, La Tronche (FR); Landry SEYVE, Saint Bonnet de Chavagne (FR); Renaud ZELLI, Le Grand Lemps (FR)

(73) Assignees: UNIVERSITE GRENOBLE ALPES, Saint Martin d'Heres (FR); CENTRE NATIONAL DE LA RECHERCHE SCIENTIFIQUE, Paris (FR); CENTRE HOSPITALIER UNIVERSITAIRE GRENOBLE ALPES, La Tronche (FR); INSTITUT POLYTECHNIQUE DE GRENOBLE, Grenoble (FR)

(21) Appl. No.: 18/016,946

(22) PCT Filed: Jul. 23, 2021
(86) PCT No.: PCT/EP2021/070656

§ 371 Date: Jan. 19, 2023

(30) Foreign Application Priority Data
Jul. 24, 2020 (FR) 2007806

(51) Int. Cl. A61K 31/192; A61P 7/04

(52) U.S. Cl. CPC A61K 31/192; A61P 7/04

(57) ABSTRACT

A compound of general formula (III′), or one of its pharmaceutically acceptable salts, for its use for the treatment of hemophilia in a subject, in particular for the restoration of coagulation in the plasma of a subject suffering from hemophilia, this compound being advantageously administered by oral route:

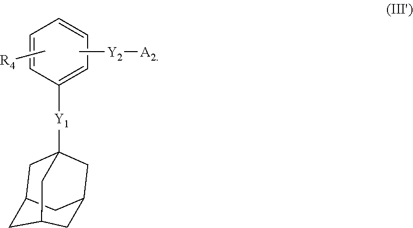

The present invention falls within the therapeutic field, more specifically in the field of the treatment of hemophilia.

More specifically, the present invention concerns a compound of particular chemical structure, for its use for the treatment of hemophilia.

Hemophilia is a rare genetic disease resulting in an impossibility for the blood to clot, and whose symptoms are spontaneous and repetitive post-traumatic bleeding in the joints and muscles. This bleeding can result in severe bleeding, whose consequences can be particularly serious.

There are two types of hemophilia: hemophilia type A, due to a deficiency of the coagulation factor FVIII, which is the most common type of hemophilia, and hemophilia type B, due to a deficiency of the coagulation factor FIX. These deficiencies in coagulation factor FVIII or FIX lead to a direct blockage of the intrinsic tenase complex, so that, to produce the factor FXa, which generates thrombin and which is essential for the formation of blood clots and therefore for coagulation, hemophiliac patients depend solely on the extrinsic pathway of coagulation, and therefore on the extrinsic tenase complex. This complex, composed of tissue factor (TF) and coagulation factor FVIIa, allows the activation of the factor FX into FXa. FXa can then be associated with the factor Va to form the prothrombinase, which accelerates the transformation of prothrombin into thrombin necessary for coagulation. However, the extrinsic tenase complex is also inhibited in hemophiliac subjects by the binding of a protein, called Tissue Factor Pathway Inhibitor (TFPI), to the FXa factor and to the complex TF-FVIIa-FXa.

TFPI is a protein that exists in two isoforms, alpha and beta. The alpha isoform contains an N-terminal end of 22 residues, followed by three Kunitz domains (K1, K2, K3) and then a long negatively charged C-terminal end. The beta isoform lacks a K3 domain and is terminated by a different C-terminal end. The three-dimensional structure of each Kunitz domain is experimentally known. In the TFPI-TF-FVIIa-FXa complex, the generally recognized hypothesis is that the K2 domain of TFPI binds to the active site of FXa and the K1 domain of TFPI binds to the active site of FVIIa, as described in particular in the publication from Girard et al., 1989, Nature 338(6215), 518-20.

At present, hemophilia is systematically treated by replacement therapies, consisting of intravenous injections, in the subject affected by the disease, of the missing factors FVIII or FIX. These treatments, in addition to their binding mode of administration, have the disadvantage of generating antibodies.

New therapeutic strategies currently under development, such as that described in the publication by Franchini et al., 2018, Blood transfusion, 16, 457-461, are based on the use of proteins which either increase coagulation, such as emicizumab, a bispecific monoclonal antibody, restoring the coagulation equivalently to 10 to 20 IU/dL (10 to 20%) of factor VIII, but which can under certain conditions be responsible for thrombotic accidents, or blocks the main anticoagulants of the coagulation cascade, in particular TFPI. The Concizumab, an anti-TFPI monoclonal antibody, has in particular been proposed as an inhibitor of TFPI.

The present invention aims to provide a treatment for hemophilia that does not in particular have the disadvantages of antibody/protein-based treatments, this treatment making it possible to effectively restore coagulation in patients affected by the disease, preferably being administered by oral route.

Additional objects of the invention are that this treatment be inexpensive and easy to produce and administer, and that it causes few or no undesirable side effects.

The present inventors have now discovered that these objectives can be achieved by particular chemical molecules, derived from adamantane, and defined by a particular general formula.

Thus, the present invention concerns a compound of formula (III′) below, or one of its pharmaceutically acceptable salts, for its use, as an active agent, for the treatment of hemophilia in a subject suffering from illness:

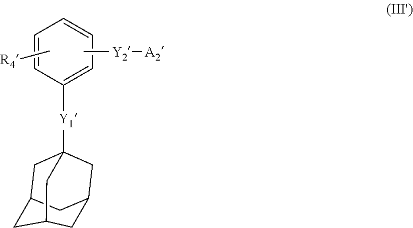

in which

Y₁′ represents a covalent bond or an amide group,

R₄′ represents a hydrogen atom, a hydroxyl group, a halogen atom, an amine group or a linear or branched, saturated or unsaturated carbon radical, which is optionally interrupted and/or substituted by one or more heteroatoms and/or one or more several groups including at least one heteroatom,

Y₂′ represents a covalent bond or an amide group,

A₂′ represents an optionally substituted cyclic or heterocyclic group including two fused rings, at least one of said rings being aromatic.

When Y₁′ represents an amide group, the nitrogen atom of this group can be linked to the adamantyl unit as well as to the phenyl radical.

Similarly, when Y₂′ represents an amide group, the nitrogen atom of this group may just as well be bonded to the phenyl radical as to the group A₂′.

In the present description, the term treatment is understood to mean a curative treatment of the bleeding episodes linked to the disease, and in particular the reduction and/or inhibition of the development of at least one of the associated symptoms, in particular the improvement of clotting and decrease in the amount and/or frequency of bleeding.

The subject treated according to the invention is in particular a mammal, for example a non-human mammal. It is preferably a human being.

The compound used according to the invention advantageously makes it possible to restore coagulation, by restoring the generation of thrombin, in the plasma of a subject suffering from hemophilia, of type A as well as of type B, including for subjects affected by severe forms of the disease.

In particular, it has been discovered by the present inventors that the compound according to the invention, subjected to an ex vivo test for the generation of fluorimetric thrombin, on the plasma of subjects suffering from severe hemophilia A, allows, at a dose of 50 μM, to restore the generation of thrombin equivalent to FVIII and even higher depending on the conditions.

The mechanisms underlying such an advantageous effect of the compound used according to the invention will not be prejudged here. It may however be thought that this effect could be due, at least in part, to an inhibition, by the compound according to the invention, in particular the compound of general formula (III′) or one of its salts, of the binding of the tissue factor pathway inhibitor (TFPI) to the coagulation factor FXa.

The compound used according to the invention does not present any toxicity for mammals. It can advantageously be administered orally, much more simply than the proteins used by the prior art, which must in turn be administered by injection.

The compound according to the invention, and its pharmaceutically acceptable salts, by their chemical nature and their low molecular weight, generally less than 5 kDa and even, for certain combinations of substituents, less than 1 kDa, or even less than 500 Da, are in particular much easier, and less expensive, to prepare than the protein/antibody compounds proposed by the prior art for the treatment of hemophilia. In this respect, the compound according to the invention can be prepared by any synthesis method conventional in itself for those skilled in the art.

In the present description, the term «pharmaceutically acceptable salt» means any salt of the compound that does not cause any adverse, allergic effect or other undesirable reaction when it is administered to the subject, in particular to a human subject.

Any non-toxic conventional salt of the compound of general formula (III′) can be used according to the invention, for example a metallic salt such as a sodium, potassium, magnesium, calcium, lithium, etc. salt. Alternatively, a salt formed from organic or inorganic acids may be used, for example salts derived from inorganic acids such as hydrochloric, hydrobromic, phosphoric, sulfuric, etc. acids, and salts derived from organic acids such as acetic, trifluoroacetic, propionic, maleic, benzoic, stearic, etc. acids. The salt can be synthesized, from the compound of general formula (III′), according to any chemical method conventional in itself.

All the properties described in the present description for the compound of general formula (III′) are also applied to its pharmaceutically acceptable salts.

The general formula (III′) above further encompasses all possible combinations of isomeric forms at the asymmetric carbons, and all mixtures of such isomeric forms. From a mixture of isomers, each particular isomer can be obtained by purification methods conventional in themselves for those skilled in the art.

Preferably, in the general formula (III′), R₄′ represents an —OR₈ group or an —O—CO—R₈ group, where R₈ represents a linear or branched, saturated or unsaturated hydrocarbon radical, in particular alkyl, including from 1 to 10 carbon atoms, optionally substituted by one or two identical or different substituents R₁₄, R₁₄′, each selected from —F, —CO₂H, —SO₃H, —P(O)(OH)₂, —P(O)(OCH₃)₂, —P(O)(OCH₂CH₃)₂, —N(CH₃)₂, —N(CH₂—CH₃)₂,

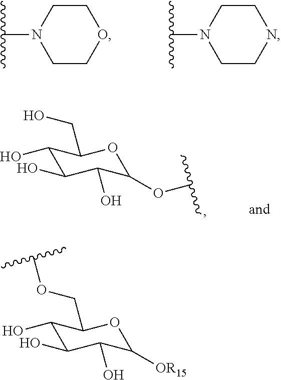

where R15 represents a hydrogen atom or a methyl group. R₈ may in particular represent a group of general formula (XVIII):

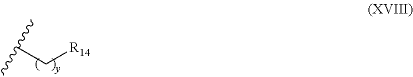

in which y is an integer comprised between 1 and 10 and R₁₄ is as defined above.

In particular embodiments of the invention, in the general formula (III′), R₄′ is fixed to the phenyl radical in the ortho or para position with respect to the adamantyl unit, and Y₂′ is fixed to the phenyl radical in the meta position relative to the adamantyl motif.

Preferably, in general formula (III′), A₂′ is at least substituted by one substituent R₁₁ selected from fluorine, carboxyl, sulphonyl, phosphonyl, tetrazole or keto-oxadiazole groups, and linear, branched and/or cyclic, saturated or unsaturated, aromatic or not carbon radicals, which are optionally interrupted and/or substituted by one or more heteroatoms, in particular fluorine, and/or one or more groups comprising at least one heteroatom, in particular carboxyl —CO₂H, sulfonyl —SO₃H and/or phosphonyl —P(O)(OH)₂.

R₁₁ can in particular be selected from tetrazole or keto-oxadiazole groups of respective formulas:

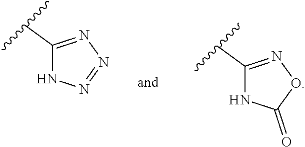 [SMILES: *c1nnn[nH]1 ; *c1noc(=O)[nH]1]

In particular embodiments of the invention, the compound used for the treatment of hemophilia corresponds to the general formula (IX):

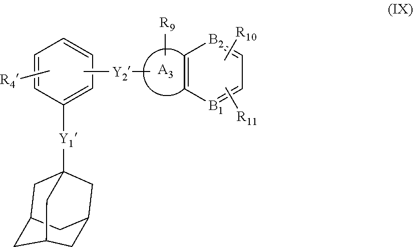

in which

Y₁′, Y₂′ and R₄′ are as defined above,

A₃ represents a cyclic or 3- to 8-membered heterocyclic, saturated or unsaturated, aromatic or not hydrocarbon, which is fused to the adjacent six-membered aromatic ring,

B₁ and B₂, which are identical or different, each represent a —CH— group or a nitrogen atom,

R₉ and R₁₀, identical or different, each represent a hydrogen atom, a hydroxyl group or an —OR₁₂ or —CO—O—R₁₂ group where R₁₂ represents a linear or branched, saturated or unsaturated hydrocarbon radical, in particular alkyl, including 1 to 10 carbon atoms, optionally substituted by one or two identical or different substituents R₁₆, R₁₆′, each selected from —F, —CO₂H, —SO₃H, —P(O)(OH)₂, —P(O)(OCH₃)₂, —P(O)(OCH₂CH₃)₂, —N(CH₃)₂, —N(CH₂—CH₃)₂,

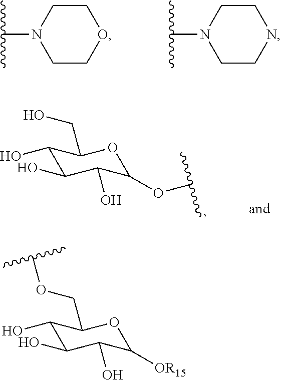

where R₁₇ represents a hydrogen atom or a methyl group,
and R₁₁ represents a substituent selected from fluorine, carboxyl, sulphonyl, phosphonyl, tetrazole or keto-oxadiazole groups, and linear, branched and/or cyclic, saturated or unsaturated, aromatic or non-aromatic carbon radicals, which are optionally interrupted and/or substituted by one or more heteroatoms, in particular fluorine, and/or one or more groups including at least one heteroatom, in particular carboxyl, sulfonyl and/or phosphonyl.

R₁₁ can in particular represent a —(CH₂)ₓ—R₁₃ group where x is an integer comprised between 0 and 4 and R₁₃ represents a fluorine atom or a carboxyl, sulphonyl, phosphonyl, tetrazole or keto-oxadiazole group, in particular a tetrazole or keto-oxadiazole group of respective formulas:

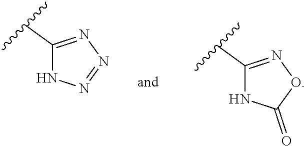 [SMILES: *c1nnn[nH]1 ; *c1noc(=O)[nH]1]

In general formula (IX), R₉ and R₁₀, which are identical or different, may also each represent a group of general formula (XVIII′):

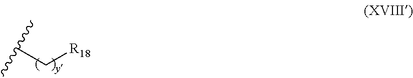

in which y′ is an integer comprised between 1 and 10 and R₁₈ is selected from —F, —CO₂H, —SO₃H, —P(O)(OH)₂, —P(O)(OCH₃)₂, —P(O)(OCH₂CH₃)₂, —N(CH₃)₂, —N(CH₂—CH₃)₂,

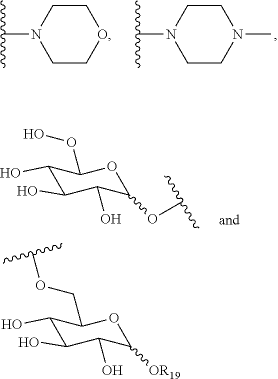

where R₁₉ represents a hydrogen atom or a methyl group.

The compound used according to the invention may in particular correspond to the general formula (X):

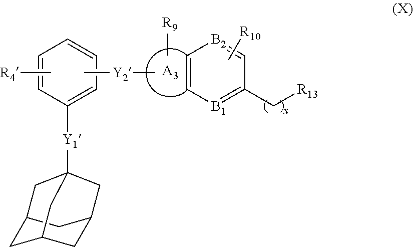

in which Y₁′, Y₂′, R₄′, A₃, B₁, B₂, R₉, R₁₀, R₁₃ and x are as defined above.

It can in particular correspond to the general formula (XI):

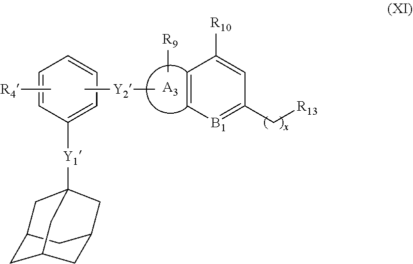

in which Y₁′, Y₂′, R₄′, A₃, B₁, R₉, R₁₀, R₁₃ and x are as defined above.

It can otherwise correspond to the general formula (XII):

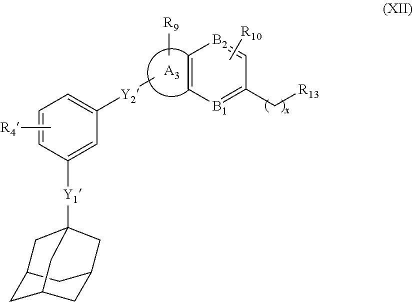

in which Y₁′, Y₂′, R₄′, A₃, B₁, B₂, R₉, R₁₀, R₁₃ and x are as defined above.

In particular embodiments of the invention, the used compound corresponds to the general formula (XIII):

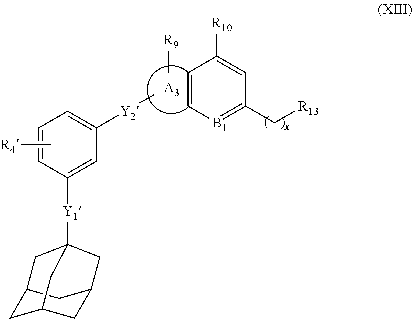

in which Y₁′, R₄′, A₃, B₁, R₉, R₁₀, R₁₃ and x are as defined above.

In particular, it can correspond to the general formula (XIV):

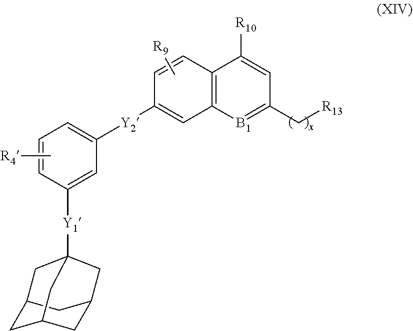

in which Y₁′, R₄′, R₉, B₁, R₁₀, R₁₃ and x are as defined above.

In particular, it can correspond to the general formula (XV):

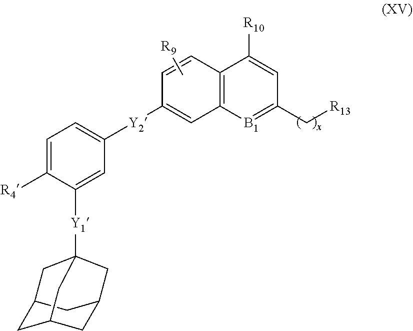

in which Y₁′, R₄′, R₉, B₁, R₁₀, R₁₃ and x are as defined above.

A particularly preferred sub-family of the general formula (XV) in the context of the invention corresponds to the general formula (XVI):

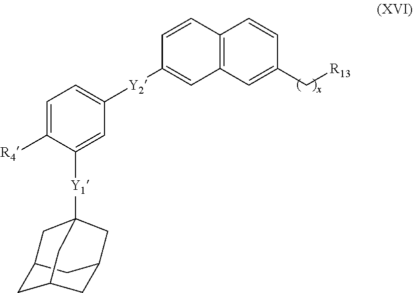

in which Y₁′, Y₂′, R₄′, R₁₃ and x are as defined above.

More generally, the present invention concerns a compound of formula (I) below, or one of its pharmaceutically acceptable salts, for its use, as an active agent, for said treatment of hemophilia in a subject suffering from illness:

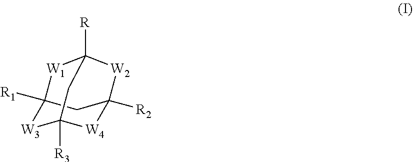

in which

W₁, W₂, W₃ and W₄, which are identical or different, each represent an oxygen atom or a bivalent radical selected from the groups —CH₂—, carbonyl —CO—, amine, in particular secondary amine —NH—, and sulphonyl —SO₂—,

R₁ and R₂, which are identical or different, each represent a hydrogen atom, a hydroxyl group or a linear, branched and/or cyclic, saturated or unsaturated, aromatic or not hydrocarbon radical, preferably C1-C8 and in particular C1-C4, which is optionally substituted, possibly comprising one or more heteroatoms and/or one or more groups including at least one heteroatom and possibly including a single cycle or several cycles, where appropriate fused cycles,

R₃ represents a hydrogen atom, a halogen atom, an alkyl group, preferably C1-C8 and preferably C1-C4, or a hydroxyl group,

and R represents a hydrogen atom, a hydroxyl group, an —NH₂ group or a linear, branched and/or cyclic, saturated or unsaturated, aromatic or not hydrocarbon radical, which is optionally substituted, which may contain one or more heteroatoms and/or a or several groups including at least one heteroatom and possibly comprising a single cycle or several cycles, where appropriate fused cycles.

The compound used according to the invention is adamantane, or tricyclo[3.3.1.1(3.7)]decane, of chemical formula:

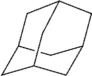

or one of its derivatives or analogues corresponding to the general formula (I).

Any non-toxic conventional salt of the compound of general formula (I) can be used according to the invention, for example a metallic salt such as a sodium, potassium, magnesium, calcium, lithium, etc. salt. Alternatively, a salt formed from organic or inorganic acids may be used, for example salts derived from inorganic acids such as hydrochloric, hydrobromic, phosphoric, sulfuric, etc. acids, and salts derived from organic acids such as acetic, trifluoroacetic, propionic, maleic, benzoic, stearic, etc. acids. The salt can be synthesized, starting from the compound of general formula (I), according to any chemical method conventional in itself.

All the properties described in the present description for the compound of general formula (I) are also applied to its pharmaceutically acceptable salts.

The general formula (I) above further encompasses all possible combinations of isomeric forms at the asymmetric carbons, and all mixtures of such isomeric forms. From a mixture of isomers, each particular isomer can be obtained by purification methods conventional in themselves for those skilled in the art.

A particularly preferred isomer according to the invention corresponds to the general formula (I′):

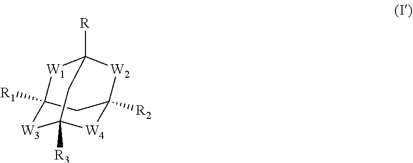

In particular embodiments, the compound used according to the invention meets one or more of the characteristics below, implemented alone or in any technically relevant combination.

Preferably, at least one, preferably at least two, preferably at least three and preferably all four, among W₁, W₂, W₃ and W₄, represent(s) a methylene —CH₂— bridge.

R₁ and R₂, which are identical or different, also preferably each represent a hydrogen atom, a hydroxyl group, a C1-C8 alkyl group, preferably a C1-C4 alkyl group or an optionally substituted phenyl radical.

R₁ and R₂ can for example be identical, and each represent a hydrogen atom or a methyl group. They can otherwise be different, and for example represent for one, a hydrogen atom, and for the other, a hydroxyl group.

Particular compounds according to the invention correspond to the following combinations of characteristics:
W₁, W₂, W₃ and W₄ each represent a methylene bridge, R₃ represents a hydrogen atom, R₁ represents a hydrogen atom and R₂ represents a hydroxyl group;
W₁, W₂, W₃ and W₄ each represent a methylene bridge, R₃ represents a hydrogen atom and R₁ and R₂ each represent a methyl group;
W₁, W₃ and W₄ each represent a methylene bridge, W₂ represents a carbonyl group, R₃ represents a hydrogen atom, and R₁ and R₂ each represent a methyl group.

W₂ can alternatively represent a group of formula:

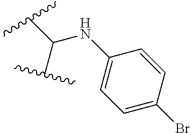 [SMILES: *C(*)Nc1ccc(Br)cc1]

In this respect, the compound of general formula (I) can be bromantane, in which W₁, W₃ and W₄ each represent a methylene bridge —CH₂—, and R₁, R₂, R₃ and R each represent a hydrogen atom.

In particular embodiments of the invention, R₂ is selected from the groups of chemical formulas:

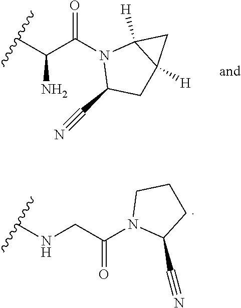 [SMILES: *[C@H](N)C(=O)N1[C@H](C#N)C[C@@H]2C[C@@H]21 ; *NCC(=O)N1CCC[C@H]1C#N]

The compound used according to the invention may thus in particular be saxagliptin, of chemical formula:

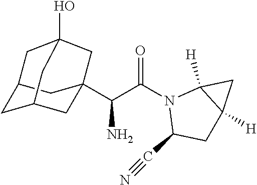 [SMILES: N#C[C@@H]1C[C@@H]2C[C@@H]2N1C(=O)[C@@H](N)C12CC3CC(CC(O)(C3)C1)C2]

or vildagliptin, of the chemical formula:

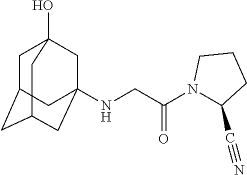 [SMILES: N#C[C@@H]1CCCN1C(=O)CNC12CC3CC(CC(O)(C3)C1)C2]

In the general formula (I), R can represent a primary amine group.

The compound used according to the invention can then in particular be amantadine or memantine, in which W₁, W₂, W₃ and W₄ each represent a methylene —CH2— bridge, R₃ represents a hydrogen atom, and R₁ and R₂ each represent a hydrogen atom for the first, and a methyl group for the second.

R may otherwise, for example, represent a group selected from the following groups:

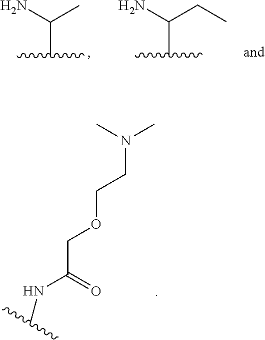

The compound used according to the invention can then in particular be adapromine, rimantadine or tromantadine, in which W₁, W₂, W₃ and W₄ each represent a methylene —CH₂— bridge, and R₁, R₂ and R₃ each represent a hydrogen.

In particularly preferred embodiments of the invention, R represents a group of formula —Y₁-A₁, in which:

Y₁ represents a covalent bond, an amine group or a linear or branched, saturated or unsaturated carbon radical, which is optionally interrupted and/or substituted by one or more heteroatoms and/or one or more groups including at least one heteroatom, said carbon radical including preferably 1 to 4 carbon atoms, in particular an —NH—CO—, —CO—NH—, —NH—CS— or —CS—NH— group,

and A₁ represents a cyclic or heterocyclic, saturated or unsaturated, optionally substituted hydrocarbon, which may include a single ring, aromatic or not, or several fused rings, each of said rings possibly being aromatic or not.

The compound then corresponds to the general formula (II):

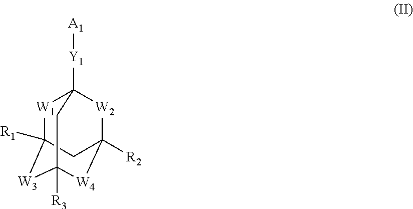

in which W₁, W₂, W₃, W₄, R₁, R₂, R₃, Y₁ and A₁ are as defined above.

A₁ can in particular be of the monocyclic, bicyclic or tricyclic type.

Preferably, A1 represents a monocyclic unit, preferably aromatic, including from 4 to 6 atoms, one or more of these atoms possibly being a heteroatom, and substituted on at least one, preferably at least two (this being understood in addition to the bond to Y₁), ring atoms.

A₁ may in particular represent a phenyl radical, substituted on at least two of the ring atoms (this being understood in addition to the bond to Y₁), the substituents preferably being located in the para position with respect to each other, one of the substituents further preferably being located in the ortho position relative to the bond Y₁.

Preferably, A₁ is substituted, in the position ortho to the bond to Y₁, by a group R₄ representing a hydrogen atom, a hydroxyl group, a halogen atom, an amine group or a linear, branched and/or cyclic, saturated or unsaturated, aromatic or not carbon radical, which is optionally interrupted and/or substituted by one or more heteroatoms and/or one or more groups including at least one heteroatom.

In particularly preferred embodiments according to the invention, in particular when A₁ represents an aromatic carbocyclic or heterocyclic group, in particular with 6 atoms, A₁ is substituted by at least one group of general formula (II′):

—Y₂-A₂ (II′)

in which

Y₂ represents a covalent bond, an amine group, or a linear or branched, saturated or unsaturated carbon radical, which is optionally interrupted and/or substituted by one or more heteroatoms and/or one or more groups including at least one heteroatom, preferably C1-C4, in particular an —NH—CO—, —CO—NH—, —NH—CS— or —CS—NH— group,

and A₂ represents an optionally substituted cyclic or heterocyclic group, which may include a single ring, which may or may not be aromatic, or several fused rings, each of said rings may or may not be aromatic.

A₂ can in particular be of the monocyclic, bicyclic or tricyclic type.

Preferably, A₂ represents an optionally substituted cyclic or heterocyclic group including two fused rings, at least one of said rings being aromatic, each of said rings preferably including between 4 and 6 atoms, one or more of these atoms possibly being a heteroatom.

Preferably, in such a configuration, each of the rings of A₂ is aromatic. Each of the rings also preferably includes 6 carbon atoms.

A₂ is preferably substituted on at least one, preferably at least two, of the atoms of at least one ring. A₂ is preferably substituted on at least one, preferably at least two, ring atoms not carrying the bond to Y₂.

Preferably, A₂ represents a naphthalene unit, optionally substituted, and preferentially substituted on at least the ring not carrying the bond to Y₂.

In particular embodiments of the invention, A₂ is substituted by at least one group selected from the group consisting of halogen atoms, in particular chlorine, bromine or iodine atoms, hydroxyl, amine or amine oxide groups, and linear, branched and/or cyclic, saturated or unsaturated, aromatic or non-aromatic carbon radicals, which are optionally interrupted and/or substituted by one or more heteroatoms and/or one or more groups including at least one heteroatom. As examples of such radicals, mention may be made of the amide, ketoxime, carbonyl, carboxyl, ester, alkyl, in particular C1-C8, in particular C1-C4, aryl, etc. radicals.

In particular embodiments of the invention, the compound corresponds to the general formula (III):

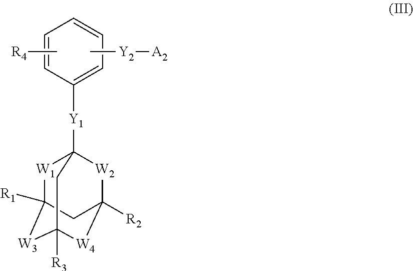

in which

W₁, W₂, W₃, W₄, R₁, R₂, R₃, Y₁, Y₂ and A₂ are as defined above,

and R₄ represents a hydrogen atom, a hydroxyl group, a halogen atom, an amine group or a linear, branched and/or cyclic, saturated or unsaturated, aromatic or non-aromatic carbon radical, which is optionally interrupted and/or substituted by a or more heteroatoms and/or one or more groups including at least one heteroatom.

In particular, in the general formula (III), R₄ can represent a hydrogen atom or an —OR5 group, where R₅ represents a C1-C8, preferably C1-C4, alkyl group, and in particular a methyl group.

The compound used according to the invention may, for example, correspond to one of the general formulas (IIIa), (IIIb), (IIIc) or (IIId) below:

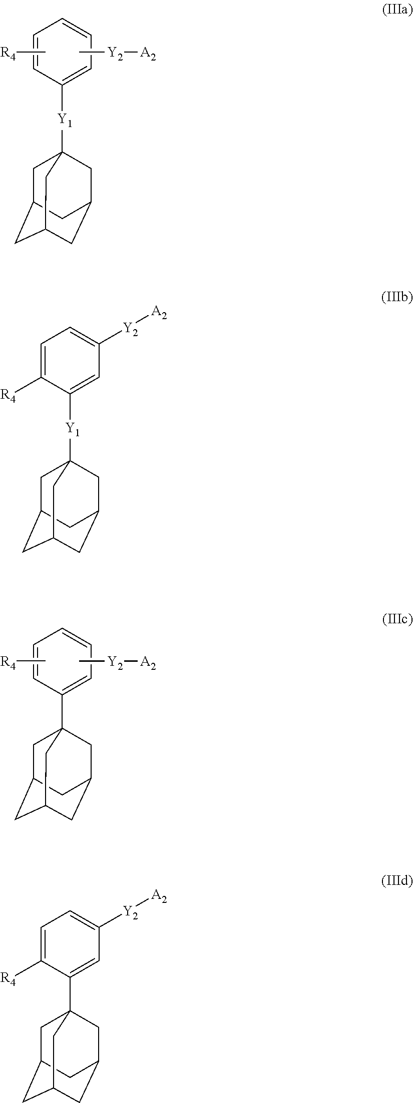

in which R₄, Y₂ and A₂ are as defined above.

In particularly preferred embodiments of the invention, the compound corresponds to the general formula (IV):

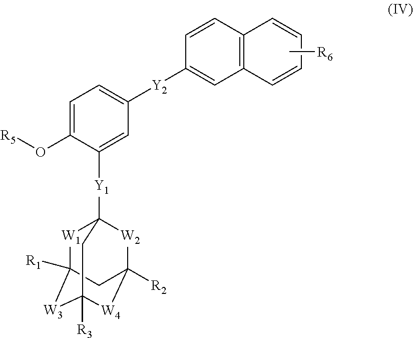

in which

W₁, W₂, W₃, W₄, R₁, R₂, R₃, Y₁ and Y₂ are as defined above,

R₅ represents a hydrogen atom or a C1-C8, preferably C1-C4 alkyl group, for example a methyl group,

and R₆ represents a halogen atom, in particular a chlorine, bromine and iodine atom, a hydroxyl, amine or amine oxide group, or a linear, branched and/or cyclic, saturated or unsaturated, aromatic or not carbon radical, which is optionally interrupted and/or substituted by one or more heteroatoms and/or one or more groups including at least one heteroatom.

R₆ can for example represent an amide, ketoxime, carbonyl, carboxyl, ester, in particular C1-C8, in particular C1-C4 alkyl, aryl, etc. radical.

In particular embodiments of the invention, R₆ represents a —CO—OR₇ group, where R₇ represents a hydrogen atom, a C1-C8, preferably C1-C4, alkyl group, an aryl group or a C6-C14 arylalkyl group.

The compound used according to the invention may in particular correspond to one of the general formulas (IVa), (IVb), (IVc) and (IVd) below:

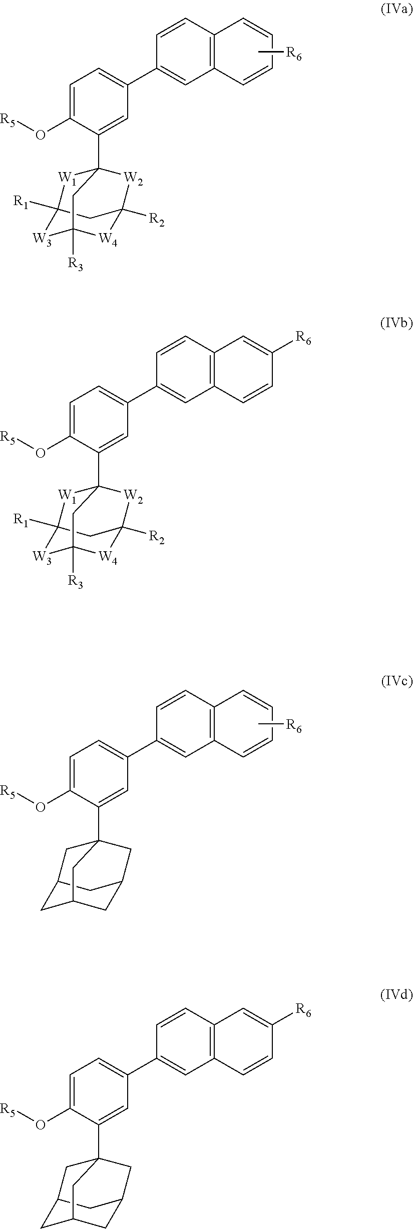

in which W₁, W₂, W₃, W₄, R₁, R₂, R₃, R₅ and R₆ are as defined above

A particularly preferred compound according to the invention is adapalene, name given to 6-[3-(1-adamantyl)-4-methoxyphenyl]naphthalene-2-carboxylic acid, of formula (V):

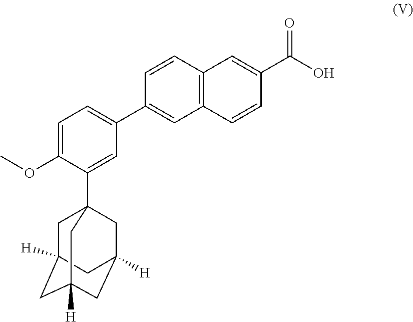 [SMILES: COc1ccc(-c2ccc3cc(C(=O)O)ccc3c2)cc1C12C[C@H]3C[C@@H](C1)C[C@@H](C2)C3]

Adapalene is particularly effective in restoring coagulation in the plasma of patients with severe hemophilia A.

Other particularly preferred compounds according to the invention, corresponding in particular to the general formula (III′), have the formulas (XVII) and (XIX):

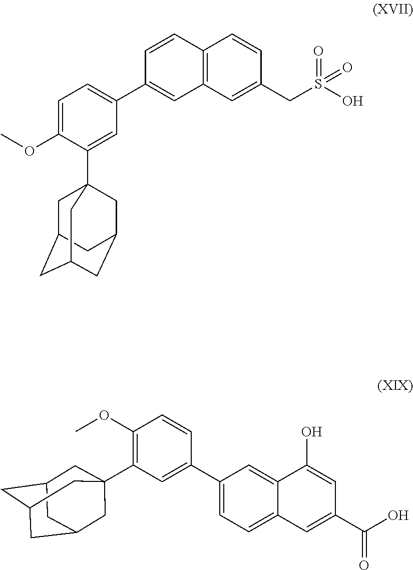

Other compounds which can be used in accordance with the invention, corresponding in particular to the general formula (III′), have the formulas (XX), (XXI), (XXII) and (XXIII):

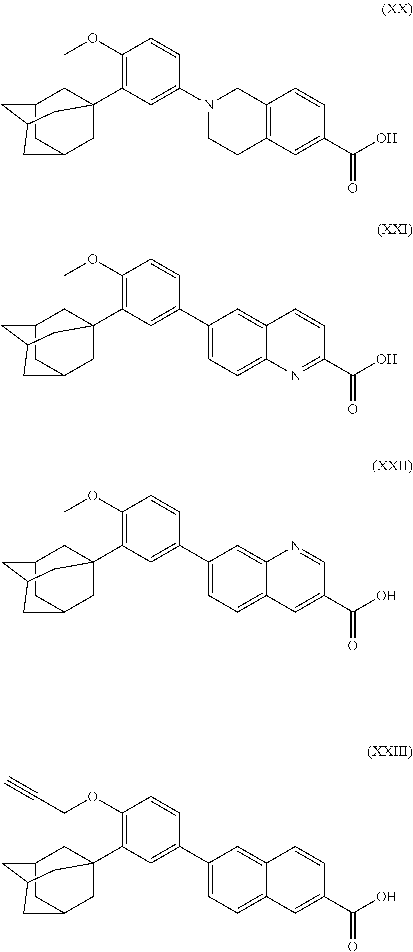

Other examples of compounds which can be used according to the invention have the formulas (VI), (VII) and (VIII) below:

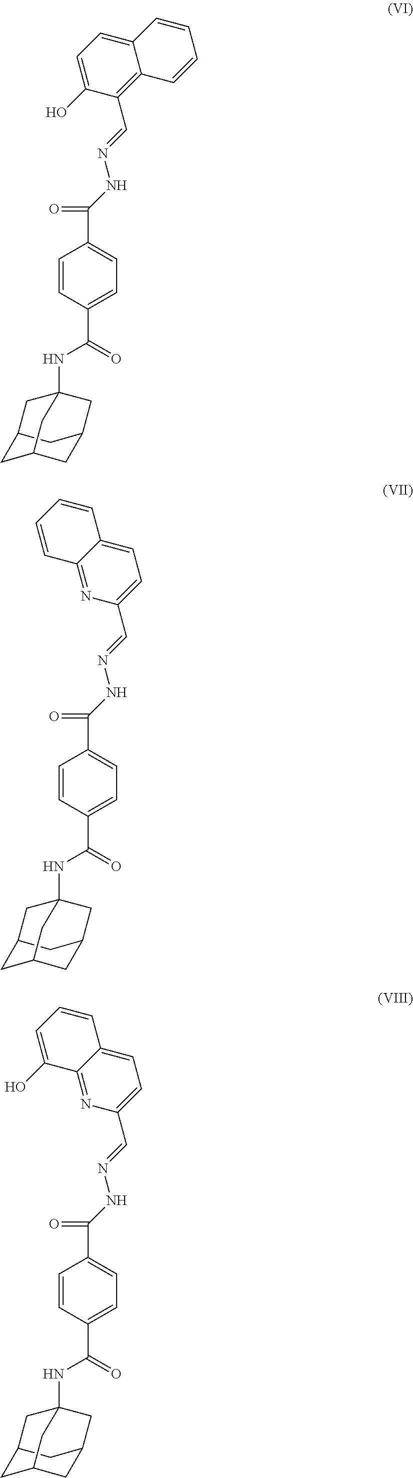

The compound according to the invention can be administered to the subject in need thereof, that is to say suffering from hemophilia, in a therapeutically effective amount, by any route, in particular by the enteral route, in particular oral, buccal or rectal, parenterally, in particular subcutaneous, intramuscular, intravenous, intradermal, etc. The administration of the compound to the treated subject is preferably carried out by oral route.

The term «therapeutically effective amount» means the amount of the compound which makes it possible, when it is administered to the subject, to obtain the desired level of therapeutic response, in particular, for the particular case of hemophilia, the level of restoration of the desired clotting. The therapeutically effective dose level of each specific compound for a particular subject varies depending on many factors such as, for example, the exact pathology and its severity, body weight, age and general health of the subject, duration of treatment, any drugs used in parallel, the sensitivity of the individual to be treated, etc. Accordingly, the optimal dosage is determined by the doctor based on the parameters that he considers relevant. The administration dosage of the compound used according to the invention can for example be taken once or twice a day.

In preferred embodiments of the invention, the compound is contained in a pharmaceutical composition, within which it constitutes an active principle, and is contained in a pharmaceutically acceptable vehicle.

This pharmaceutical composition may be in any form suitable for enteral or parenteral administration. It is preferably presented in a form suitable for administration to the subject by the oral route.

All of the constituents of this pharmaceutical composition are of course selected to be pharmaceutically compatible, that is to say that they do not produce any adverse, allergic or other undesirable reaction when they are administered to the subject, in particular to a mammal and in particular to a human.

The pharmaceutical composition may contain any conventional excipient by itself. Such an excipient can be a diluent, an adjuvant or any other conventional substance in itself for the constitution of medicaments, such as a preservative, filler, disintegrating, wetting, emulsifying, dispersing, antibacterial or antifungal agent, etc., or any mixture thereof

It may further contain one or more other active ingredients, acting or not in synergy with the compound used according to the invention,

The pharmaceutical composition can be formulated according to any pharmaceutical form suitable for oral administration in mammals, and in particular in humans. It may in particular be in the form of a powder, of tablets, of capsules, of granules, of a syrup, or of an oral solution or suspension, prepared in a conventional manner by itself.

It is preferably packaged in the form of unit doses, each dose containing a therapeutically effective amount of the compound according to the invention. The concentration of the compound in each dose of the pharmaceutical composition is thus preferably selected to deliver to the subject, at each administration, an amount of compound which is effective to obtain the desired therapeutic response. The pharmaceutical composition is for example packaged in the form of unit doses, each may in particular comprise an amount comprised between 1 and 10 g of the compound according to the invention.

The present invention is also expressed in terms of a method for treating hemophilia in a subject, and in particular a method for restoring coagulation in the plasma of a subject afflicted with hemophilia. The subject can in particular be a mammal, and preferentially a human being. This method comprises the administration, to said subject in need, of a therapeutically effective amount of the compound as defined above, or one of its pharmaceutically acceptable salts. This method can meet one or more of the characteristics described above with reference to the use of the compound according to the invention for the treatment of hemophilia.

The present invention is also expressed in terms of the use of a compound according to the invention, or of one of its pharmaceutically acceptable salts, for the manufacture of a medicament for the treatment of hemophilia. This use may correspond to one or more of the characteristics described above with reference to the use of the compound according to the invention for the treatment of hemophilia.

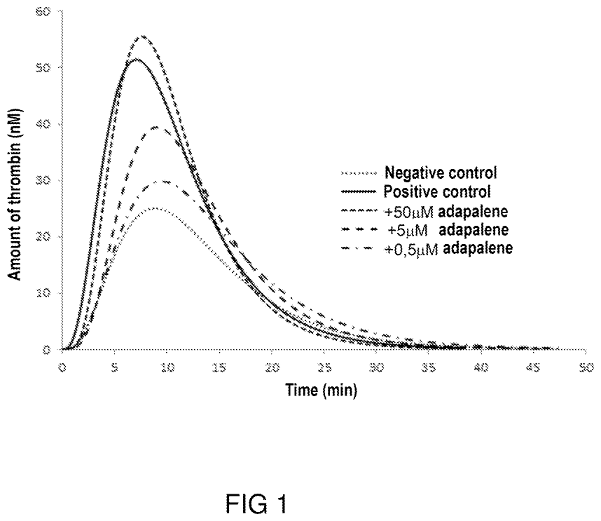

The characteristics and advantages of the invention will appear more clearly in the light of the examples of implementation below, provided purely by way of illustration and in no way limiting of the invention, with the assistance of FIGS. 1 to 4, in which:

FIG. 1 shows a graph representing the results (amount of thrombin as a function of time) of an ex vivo thrombin generation test for the plasma of a subject suffering from severe hemophilia A, in the presence of plasma factor FVIII (100% VIII, positive control), dilution buffer (0% VIII, negative control), and a compound according to the invention, adapalene, at respective concentrations of 0.5 μM, 5 μM and 50 μM.

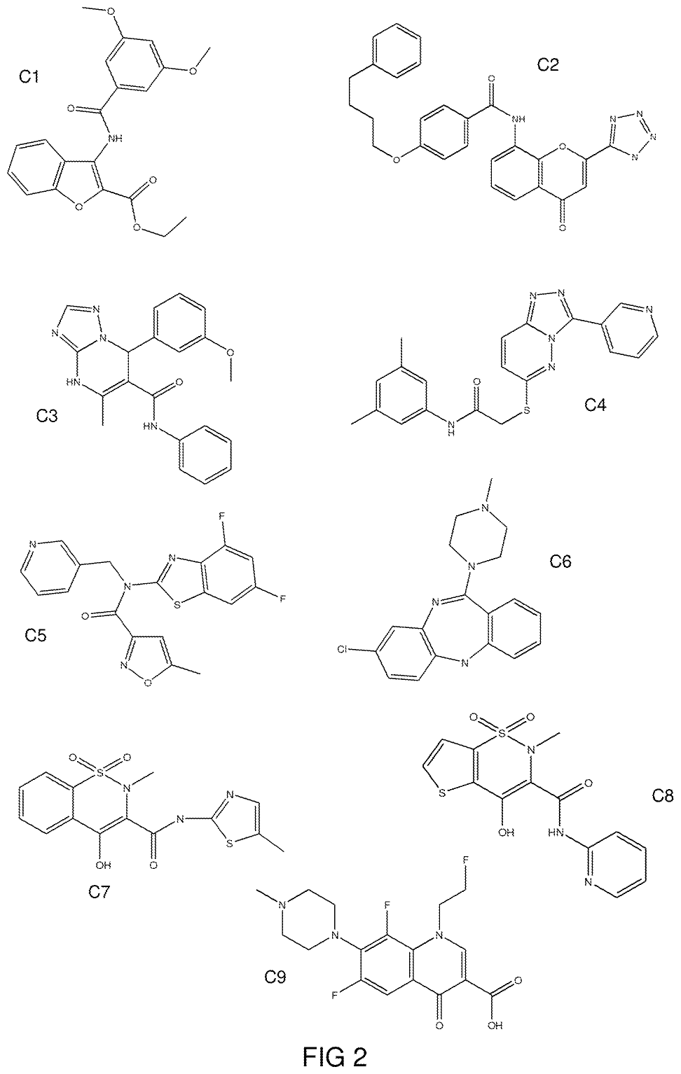

FIG. 2 represents the chemical structure of comparative compounds C1 to C9 implemented in an example, not corresponding to the general formula (I).

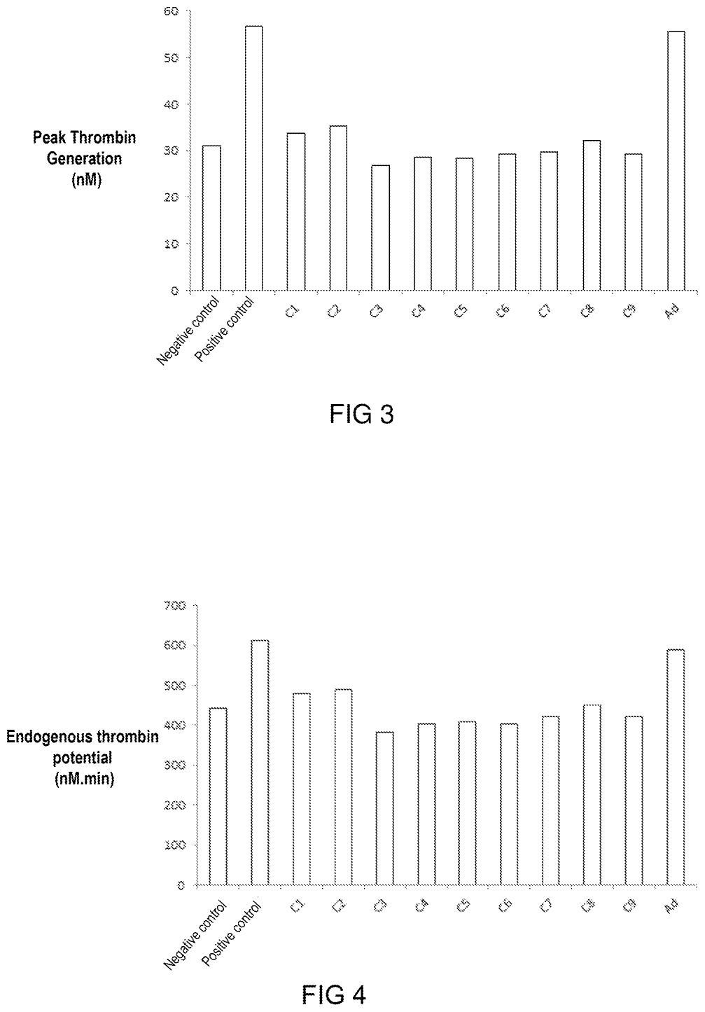

FIG. 3 shows a histogram representing the amount of thrombin measured at the peak during an ex vivo thrombin generation test (TGT) in the plasma of an individual suffering from severe hemophilia A, in the presence of plasma factor FVIII (100% VIII, positive control), dilution buffer (0% VIII, negative control) and in the presence of adapalene at 50 μM (Ad) or of a comparative compound C1 to C9 not corresponding to the general formula (I).

FIG. 4 shows a histogram representing the total amount of thrombin generated (endogenous thrombin potential) during an ex vivo thrombin generation test (TGT) in the plasma of an individual with severe hemophilia A, in the presence of plasma FVIII factor (100% VIII, positive control), dilution buffer (0% VIII, negative control) and in the presence of adapalene at 50 μM (Ad) or a comparative compound C1 to C9 not corresponding to the general formula (I).

### Example 1—Ex Vivo Thrombin Generation Test

When coagulation is activated, a chain of enzymatic reactions, called the coagulation cascade, occurs. It results in the production of thrombin (factor IIa), the last enzymatic «link» in this coagulation cascade.

The thrombin generation test consists of measuring the kinetics of appearance of this key coagulation factor in the plasma over time. After activation of coagulation by calcium, the amount of thrombin changes over time. It is measured using a synthetic thrombin substrate coupled to a fluorescent molecule (ZGGR-AMC). The main parameters determined are the latency time before observing an increase in thrombin generation, the thrombin peak corresponding to the maximum amount of thrombin generated and the ETP (endogenous thrombin potential) corresponding to the total amount of thrombin generated in plasma during the test. In the case of a patient with hemophilia A, the generation of thrombin is low or even almost zero for some patients. Adding the missing coagulation factor (factor VIII) to its physiological level (1 Ul/mL or 100%) restores coagulation in patients and recovers a «normal» thrombin generation. Thus, the thrombin generation test makes it possible to globally evaluate the coagulation rate of a given plasma and in the present case, it makes it possible to evaluate the capacity of a molecule to restore or not restore coagulation in a hemophiliac patient.

For this example, several ex vivo thrombin generation tests were carried out on plasma from a patient suffering from severe hemophilia A, therefore devoid of factor FVIII, in order to evaluate the effect on coagulation of a compound of general formula (I), more particularly adapalene, of formula (V) above, on the kinetics and the generated amount of thrombin.

Hemophiliac patient plasma was obtained from Cryopep company (Montpellier, France).

Adapalene is commercially available, in particular from Prestwick company. It was dissolved in an 8% dimethylsulfoxide (DMSO) solution then diluted to a concentration of 1 mM and then tested at the following concentrations: 50 μM, 5 μM, 0.5 μM (final concentrations in plasma).

The thrombin generation test was performed at 37° C. using a fully automated STA-Genesia analyzer (Diagnostica Stago) with STG®-Bleedscreen reagent (Diagnostica Stago) according to the manufacturer's instructions and according to the method established by Pr. Hemker in 2003. The STG®-Bleedscreen reagent is a mixture of tissue factor (TF) at low concentration with phospholipid vesicles (PL). The test is triggered by the addition of a mixture of calcium+fluorescent substrate (ZGGR-AMC) (STG®-FluoStart mixture).

From a technical point of view, 4 volumes of surcharged plasma sample are added to 1 volume of «initiator complex» (STG®-Bleedscreen) and incubated for 10 min at 37° C. Then, the reaction is triggered by adding 1 volume of STG®-FluoStart (mixture of CaCl₂ and ZGGR-AMC substrate) and the fluorescence signal is measured over time. Each test is performed in duplicate

For each tested molecule, 475 μL of plasma from the hemophiliac patient were surcharged with 25 μL of a solution containing different amounts of compound (molecule to be tested, factor VIII or dilution buffer) to obtain the desired final concentration (systematic dilution of the molecule to be tested in the plasma at 1/20). For example, to obtain the final concentration of 50 μM in plasma, 25 μL of a 1 mM solution was added to 475 μL of plasma. The preparation of 500 μL of surcharged plasma allows to perform the 2 thrombin generation tests (duplicate), the 2 calibration tests (duplicate) and takes into account the dead volume of the automaton.

In parallel with the tests on the different chemical compounds, the plasma of a hemophiliac patient is surcharged with the same buffer for diluting the compound, an 8% solution of dimethylsulfoxide (DMSO), as a negative control (0.4% of final DMSO) in order to obtain the basal level of thrombin generation of the used plasma. The positive control is the same plasma surcharged with plasma factor VIII (Factane, LFB, France) at a concentration of 1 IU/mL (or 100% factor VIII) in order to obtain the expected «normal» level of used thrombin generation plasma.

The results obtained in thrombin generation, for each of the tested adapalene concentrations, are shown in FIG. 1.

The negative control (plasma from a hemophiliac A patient surcharged with the dilution buffer) shows weak thrombin generation with a peak at 25 nM of thrombin and an ETP at 360 nM·min. The positive control (plasma from a hemophiliac A patient surcharged with 1 IU/mL of factor VIII) shows a «normal» generation of thrombin with a peak at 51 nM and an ETP at 599 nM·min. For the different concentrations of adapalene, an increase in the generation of thrombin is observed depending on the concentration of adapalene: the higher the concentration, the greater the generation of thrombin. At 0.5 μM, adapalene already has an effect on increasing thrombin generation with a peak at 29.7 nM and an ETP at 455 nM·min. At 5 μM, the increase in thrombin generation is greater with a peak at 39 nM and an ETP at 504 nM·min. At the concentration of 50 μM, adapalene restores the generation of thrombin to the same level as the positive control (100% FVIII) with a peak at 55 nM and an ETP at 588 nM·min.

By way of comparison, several compounds with a close structure, but not corresponding to the general formula (I) (compounds C1 to C9), were also subjected to the same test. These compounds are shown in FIG. 2. Each of these compounds was surcharged into the plasma of the hemophiliac patient under the same conditions as adapalene, to the tune of 50 μM.

The results obtained, for adapalene (Ad) at 50 μM and for the comparative compounds C1 to C9, are shown in FIG. 3 and FIG. 4.

It is observed that among the various tested compounds, only adapalene (Ad) at 50 μM makes it possible to obtain an increase in the generation of thrombin compared to the negative control, with a peak of generated thrombin and an ETP at the same level as the positive control.

### Example 2—Effect on FXa Inhibition by TFPI

The ability of adapalene to reverse FXa inhibition by TFPI was assessed by using a low TFPI concentration FXa activity assay. For this test, a truncated human TFPI (TFPI-K1K2) was used, which is common to both TFPI isoforms α and β, having the amino acid sequence SEQ ID No 1:

DSEEDEEHTIITDTELPPLKLMHSGCAFKADDGPCKAIMKRFFFNIFTR
QCEEFIYGGCEGNQNRFESLEECKKMCTRDNANRIIKTTLQQEKPDFCF
LEEDPGICRGYITRYFYNNQTKQCERFKYGGCLGNMNNFETLEECKNIC
EDGPNGF.

Adapalene was tested for its ability to release FXa inhibition by TFPI-K1K2 using a colorimetric assay in a 96-well plate format. All steps were performed at room temperature. FXa inhibition by increasing TFPI-K1K2 concentrations was first analyzed to determine the best detection conditions. Concentrations of TFPI-K1K2 and FXa (New England Biolabs) of 30 nM and 0.5 nM, respectively, were selected to ensure complete inhibition of FXa, without a significant excess of TFPI-K1K2. Each protein was diluted using the same buffer: 20 mM Hepes, 135 mM NaCl, 1% BSA, 2 mM CaCl2, pH 7.3. The test was performed manually in 96-well plates (Nunc Maxisorp®). The experiment was performed in duplicate at a final adapalene concentration of 50 μM.

Briefly, 14.5 μL of 350 μM adapalene, prediluted in the buffer described above, was added to 62.5 μL 50 nM TFPI-K1K2 and mixed together by aspiration/dispensing. After 10 min of incubation at room temperature, 6.25 μL of 8 nM FXa was added, the mixture was mixed again, and the activity of uninhibited FXa was quantified by the addition of 18 μL of PNAPEP-1025 (Cryopep), an FXa substrate, diluted to 2 mM in H₂O.

For the negative control, 14.5 μl of 0.25% DMSO was added to 62.5 μl of TFPI-K1K2, and for the positive control, 14.5 μl of 0.25% DMSO was added to 62, 5 μl of buffer. Then, after 10 min of incubation at room temperature, 6.25 μl of 8 nM FXa and 18 μl of PNAPEP-1025 were added as previously described.

The plates were centrifuged to remove any bubbles and the optical density (OD) at 405 nm, corresponding to the hydrolysis of the PNAPEP-1025 substrate, was measured for 1 h at room temperature. The absorbance value at t0, corresponding to an absorbance of 0.05, was subtracted and the thrombin generation restoration value was reported as a percentage of the positive control.

The following results were obtained: negative control: OD=0.048; positive control: OD=0.406; adapalene at 50 μM: OD=0.105. The measured coagulation restoration percentage is approximately 14%. This result demonstrates that adapalene exhibits an inhibition lifting effect of FXa by TFPI.

### Example 3—Synthesis of Compounds According to the Invention

### Compound H27

The compound H27 (HEMO-027) of formula:

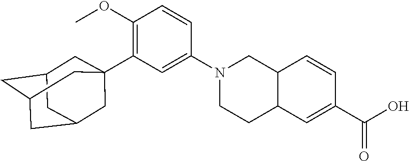 [SMILES: COc1ccc(N2CCC3C=C(C(=O)O)C=CC3C2)cc1C12CC3CC(CC(C3)C1)C2]

is prepared according to the following reaction scheme:

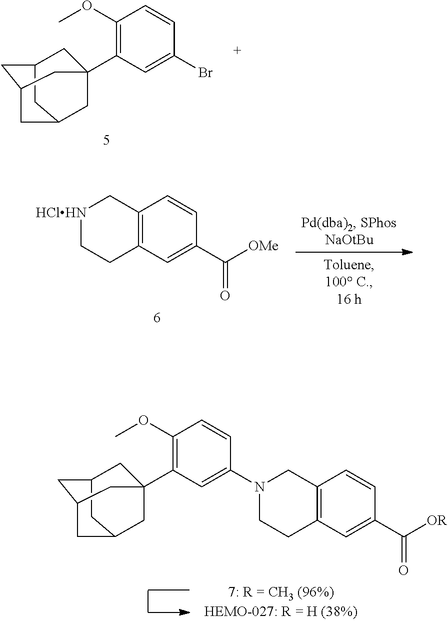

### Methyl 2-(3-(adamantan-1-yl)-4-methoxyphenyl)-1,2,3,4-tetrahydroisoquinoline-6-carboxylate (7)

Pd₂(dba)₂ (1.7 mg, 0.0016 mmol, 1 mol %) was added to a flame-dried reactor followed by SPHOS (2.6 mg, 0.0064 mmol, 4 mol %), 1-(5-bromo-2-methoxyphenyl)adamantane (5) (50 mg, 0.16 mmol, 1 eq.), methyl 1,2,3,4-tetrahydroisoquinoline-6-carboxylate hydrochloride (6) (43 mg , 0.19 mmol, 1.2 eq.) and NaOtBu (36.5 mg, 0.38 mmol, 2.4 eq.). Anhydrous toluene (600 μl, 267 mM) was added and the reactor was sealed. The resulting mixture was stirred at 100° C. for 16 h. After reaching room temperature, H₂O and AcOEt were added. The aqueous phase was extracted 3 times with AcOEt, and the combined organic phases were evaporated over MgSO₄, filtered and concentrated in vacuo. The residue was eluted on a column of silica gel with 4:1 cyclohexane-AcOEt to obtain (7) (66 mg, 96%) as a yellow solid. ¹H NMR (CDCl₃, 400 MHz): δ 7.85; (s, 1H, H-ar), 7.84; (d, 1H, J=7.9 Hz, H-ar), 7.20; (d, 1H, J=7.9 Hz, H- ar), 6.99; (s, 1H, H-ar), 6.85-6.80; (m, 2H, 2 H-ar), 4.32; (s, 2H, NCH₂), 3.91; (s, 3H, OCH₃), 3.81; (s, 3H, OCH₃), 3.45; (t, 2H, J=5.8 Hz, NCH₂CH₂), 3.05; (t, 2H, J=5.8 Hz, NCH₂CH₂), 2.11; (db, 6H), 2.07; (db, 3H), 1.78; (db, 6H). 13C NMR (CDCL₃, 100.6 MHz) δ 167.3, 153.5, 145.0, 140.4, 139.6, 135.0, 130.2, 128.3, 127.1, 126.8, 117.4, 114.7, 112.7, 55.6, 53.4, 52.1, 48.8, 40.8, 29.3.

### 2-(3-(adamantan-1-yl)-4-methoxyphenyl)-1,2,3,4-tetrahydroisoquinoline-6 carboxylic acid (HEMO-027)

To a solution of (7) (22 mg, 0.051 mmol) in 2:1 THF:H₂O (2 mL) at room temperature was added LiOH (3.5 mg, 0.13 mmol, 2.5 eq.). After stirring for 16 h, 1M HCl was added to reach a pH=1. The precipitate formed was filtered and washed with H₂O then with cold MeOH. The resulting solid was dried in vacuo to obtain (HEMO-027) (8 mg, 38%) as a light yellow solid. ¹H NMR (DMSO-d₆, 400 MHz): d 12.79; (bs, 1H, OH), 7.75; (s, 1H, H-ar), 7.73; (d, 1H, J=8.0 Hz, H-ar), 7.32; (d, 1H, J=8.0 Hz, H-ar), 6.88-6.81; (m, 3H, 3 H-ar), 4.29; (s, 2H, NCH₂), 3.73; (s, 3H, OCH₃), 3.39; (t, 2H, J=5.8 Hz, NCH₂CH₂), 2.97; (t, 2H, J=5.8 Hz, NCH₂CH₂), 2.04; (bs, 9H), 1.73; (bs, 6H). ¹³C NMR (DMSO-d₆, 125.7 MHz) d 167.3, 152.3, 144.5, 140.0, 138.2, 134.7, 130.2, 129.6, 128.6, 126.9, 126.6, 115.6, 114.2, 113.0, 55.6, 51.9, 47.5, 36.6, 28.5, 28.4. HRMS (ESI/Q-TDE) m/z calc. for C₂₇H₃₁NO₃ [M+H]⁺ 418.2377, obtained 418.2369.

### Compound H31

The compound H31 (HEMO-031) of formula:

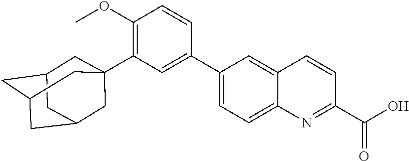 [SMILES: COc1ccc(-c2ccc3nc(C(=O)O)ccc3c2)cc1C12CC3CC(CC(C3)C1)C2]

is prepared according to the following reaction scheme:

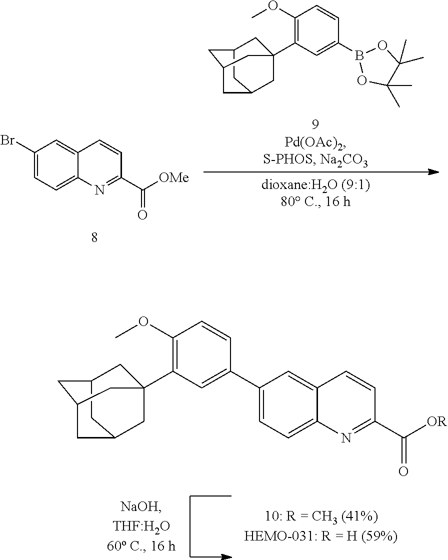

### Methyl 6-(3-(adamantan-1-yl)-4-methoxyphenyl)quinoline-2-carboxylate (10)

Pd(OAc)₂ (6.10 mg, 0.027 mmol, 5 mol %) and S-Phos (11.2 mg, 0.027 mmol, 5 mol %) were added to a degassed solution of methyl 6-bromoquinoline-2-carboxylate (8) (144.5 mg, 0.54 mmol, 1 eq.), boronic ester (9) (200 mg, 0.54 mmol, 1 eq.) and Na₂CO₃ (173 mg, 1.63 mmol, 3 eq.) in 10:1 Dioxane:H₂O. The obtained yellow suspension was stirred at 80° C. for 16 h. H₂O was then added and the aqueous phase was extracted 3 times with AcOEt. The combined organic phases were dried over Na₂SO₄, filtered and concentrated in vacuo. The residue was eluted on a silica gel column with 0 to 70% AcOEt in Cyclohexane to obtain (10) (95 mg, 41%) as a white solid. ¹H NMR (CDCl₃, 400 MHz): d 8.46; (d, 1H, J=8.9 Hz, H-ar), 8.34; (d, 1H, J=8.5 Hz, H-ar), 8.17; (d, 1H, J=8.5 Hz, H-ar), 8.03; (dd, 1H, J=2.1 Hz, J=8.9 Hz, H-ar), 7.95; (d, 1H, J=2.1 Hz, H-ar), 7.54; (d, 1H, J=2.4 Hz, H-ar), 8.03; (dd, 1H, J=2.4 Hz, J=8.4 Hz, H-ar), 6.94; (d, 1H, J=8.5 Hz, H-ar), 4.05; (s, 3H, CH₃), 3.85; (s, 3H, CH₃), 2.11; (bs, 6H), 2.04; (bs, 3H), 1.74; (bs, 6H).

### 6-(3-(adamantan-1-yl)-4-m2thoxyph2nyl)quinoline-2-carboxylic acid (HEMO-031)

To a solution of ester (10) (30 mg, 0.07 mmol, 1 eq.) in 4:1 THF:MeOH at room temperature was added 2M NaOH (105 μl, 0.21 mmol, 3 eq.). The obtained solution was mixed for 16 h at 60° C. The reaction mixture was then poured into water and the aqueous phase was extracted with AcOEt. The resulting aqueous phase was acidified using 1M HCl and extracted 3 times with AcOEt. The combined organic phases were dried over Na₂SO₄, filtered and concentrated in vacuo to obtain (HEMO-031) (17 mg, 59%) as a white solid. ¹H NMR (DMSO-d₆, 500 MHz): □ 8.57; (d, 1H, J=8.6 Hz, H-ar), 8.30; (d, 1H, J=1.4 Hz, H-ar), 8.19; (d, 1H, J=8.9 Hz, H-ar), 8.15; (dd, 1H, J=1.9 Hz, J=9.0 Hz, H-ar), 8.11; (d, 1H, J=8.5 Hz, H-ar), 7.69; (dd, 1H, J=2.2 Hz, J=8.4 Hz, H-ar), 7.61; (d, 1H, J=2.2 Hz, H-ar), 7.14; (d, 1H, J=8.6 Hz, H-ar), 3.88; (s, 3H, CH₃), 2.14; (bs, 6H), 2.08; (bs, 3H), 1.77; (bs, 6H). ¹³C NMR (DMSO-d₆, 125.7 MHz) □ 166.5, 158.8, 145.8, 140.0, 138.1, 137.4, 130.9, 130.1, 129.5, 129.2, 125.9, 125.2, 124.0, 121.1, 112.8, 55.4, 36.6, 36.5, 28.4. HRMS (ESI/Q-TDE) m/z calc. for C₂₇H₂₈NO₃ [M+H]⁺ 414.2064, obtained 414.2060.

### Compound H32

The compound H32 (HEMO-032) of formula:

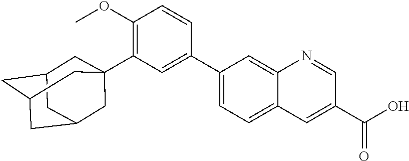 [SMILES: COc1ccc(-c2ccc3cc(C(=O)O)cnc3c2)cc1C12CC3CC(CC(C3)C1)C2]

is prepared according to the following reaction scheme:

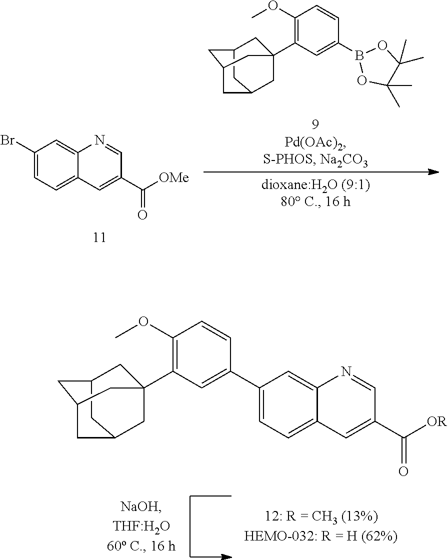

### Methyl 7-(3-(adamantan-1-yl)-4-methoxyphenyl)quinoline-3-carboxylate (12)

Pd(OAc)₂ (16.9 mg, 0.075 mmol, 10 mol %) and S-Phos (15.4 mg, 0.037 mmol, 5 mol %) were added to a degassed solution of methyl 7-bromoquinoline-3-carboxylate (11) (200 mg, 0.75 mmol, 1 eq.), boronic ester (9) (415 mg, 1.13 mmol, 1.5 eq.) and Na₂CO₃ (239 mg, 02.25 mmol, 3 eq.) in 10:1 Dioxane:H₂O. The obtained yellow suspension was stirred at 80° C. for 16 h. H₂O was then added and the aqueous phase was extracted 3 times with AcOEt. The combined organic phases were dried over Na₂SO₄, filtered and concentrated in vacuo. The residue was eluted on a silica gel column with 0 to 70% AcOEt in Cyclohexane to obtain (12) (40 mg, 13%) as a white solid. ¹H NMR (DMSO-d₆, 400 MHz): □ 9.33; (d, 1H, J=2.2 Hz, H-ar), 9.02; (d, 1H, J=2.2 Hz, H-ar), 8.28-8.26; (m, 2H, 2 H-ar), 8.05; (dd, 1H, J=1.8 Hz, J=8.7 Hz, H-ar), 7.74; (dd, 1H, J=2.3 Hz, J=8.5 Hz, H-ar), 7.63; (d, 1H, J=2.4 Hz, H-ar), 7.16; (d, 1H, J=8.6 Hz, H-ar), 3.97; (s, 3H, CH₃), 3.89; (s, 3H, CH₃), 2.16; (bs, 6H), 2.08; (bs, 3H), 1.77; (bs, 6H).

### 7-(3-(adamantan-1-yl)-4-methoxyphenyl)quinoline-3-carboxylic acid (HEMO-032)

To a solution of ester (12) (15 mg, 0.035 mmol, 1 eq.) in 2:1 THF:MeOH at room temperature, was added 2M NaOH (53 μl, 0.11 mmol, 3 eq.). The solution obtained was stirred for 2 hours at 60° C. The reaction mixture was poured into water and the aqueous phase was extracted with AcOEt. The resulting aqueous phase was acidified with 1M HCl and extracted 3 times with AcOEt. The combined organic phases were dried over Na₂SO₄, filtered and concentrated in vacuo to obtain (HEMO-032) (9 mg, 62%) as a white solid. ¹H NMR (DMSO-d₆, 500 MHz): □ 13.45; (s, 1H, OH), 9.31; (d, 1H, J=2.1 Hz, H-ar), 8.96; (d, 1H, J=1.8 Hz, H-ar), 8.25; (s, 1H, H-ar), 8.23; (d, 1H, J=8.6 Hz, H-ar), 8.02; (dd, 1H, J=1.7 Hz, J=8.5 Hz, H-ar), 7.73; (dd, 1H, J=2.2 Hz, J=8.5 Hz, H-ar), 7.61; (d, 1H, J=2.3 Hz, H-ar), 7.14; (d, 1H, J=8.6 Hz, H-ar), 3.88; (s, 3H, CH₃), 2.14; (bs, 6H), 2.07; (bs, 3H), 1.76; (bs, 6H). ¹³C NMR (DMSO-d₆, 125.7 MHz) □ 166.4, 159.0, 150.3, 149.7, 143.9, 138.2, 138.1, 130.8, 130.1, 126.6, 126.1, 125.3, 124.8, 123.1, 112.8, 55.4, 36.6, 36.5, 28.4. HRMS (ESI/Q-TDE) m/z calc. C₂₇H₂₈NO₃ [M+H]⁺ 414.2064, obtained 414.2058.

### Compound H35

The compound H35 (HEMO-035) of formula:

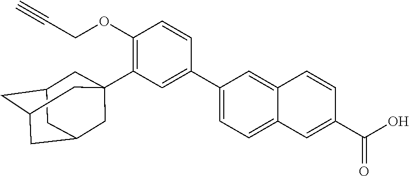 [SMILES: C#CCOc1ccc(-c2ccc3cc(C(=O)O)ccc3c2)cc1C12CC3CC(CC(C3)C1)C2]

is prepared according to the following reaction scheme:

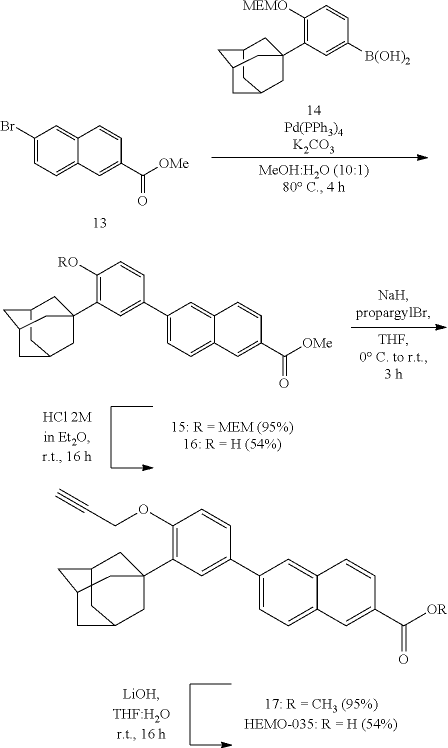

### Methyl 6-(3-(adamantan-1-yl)-4-((2-methoxyethoxy)methoxy)phenyl)-2-naphthoate (15)

Boronic acid (14) (500 mg, 1.39 mmol, 1 eq.) was added to the flame-dried reactor followed by methyl 6-bromo-2-naphthoate (13) (368 mg, 1.39 mmol, 1 eq.), Pd(PPh₃)₄ (80 mg, 0.070 mmol, 5 mol %), and K₂CO₃ (383 mg, 2.78 mmol, 2 eq.). After addition of 10:1 MeOH:H₂O (11.5 mL, 120 mM) the reactor was then sealed. The solution obtained was stirred at 80° C. for 4 hours. After reaching room temperature, H₂O and CH₂Cl₂ were added. The aqueous phase was extracted 3 times with CH₂Cl₂, and the combined organic phases were washed with saturated NaCl solution, dried over MgSO₄, filtered and concentrated in vacuo. The residue was eluted on a column of silica gel containing 0 to 5% AcOEt in toluene to obtain (15) (610 mg, 88%) in the form of a white solid. ¹H NMR (CDCl₃, 400 MHz): □ 8.61; (s, 1H, H-ar), 8.07; (dd, 1H, J=1.4 Hz, J=8.6 Hz, H-ar), 8.01; (s, 1H, H-ar), 7.99; (d, 1H, J=8.6 Hz, H-ar), 7.92; (d, 1H, J=8.6 Hz, H-ar), 7.79; (dd, 1H, J=1.6 Hz, J=8.5 Hz, H-ar), 7.92; (d, 1H, J=2.2 Hz, H-ar), 7.51; (dd, 1H, J=2.2 Hz, J=8.5 Hz, H-ar), 7.29; (d, 1H, J=8.6 Hz, H-ar), 5.39; (s, 2H, OCH₂O), 3.99; (s, 3H, OCH₃), 3.90; (dd, 1H, J=3.9 Hz, J=5.4 Hz, OCH₂), 3.62; (dd, 1H, J=3.9 Hz, J=5.4 Hz, OCH₂), 3.42; (s, 3H, OCH₃), 2.20; (s, 6H), 2.11; (s, 3H), 1.81; (s, 6H). ¹³C NMR (CDCl₃, 100.6 MHz) □ 167.4, 156.7, 141.4, 139.1, 136.0, 133.8, 131.4, 130.9, 129.8, 128.4, 127.1, 126.6, 126.2, 126.0, 125.7, 125.0, 115.2, 93.5, 71.7, 68.0, 59.2, 52.3, 40.9, 37.4, 37.2, 29.2. HRMS (ESI/Q-TDE) m/z calc. pour C₃₂H₄₀NO₅ [M+NH₄]⁺ 518.2901, obtained 518.2890.

### Methyl 6-(3-(adamantan-1-yl)-4-hydroxyphenyl)-2-naphthoate (16)

A suspension of (15) (610 mg, 1.22 mmol, 1 eq.) in HCl (2M in Et₂O, 25 mL) was stirred at room temperature for 16 h. A saturated solution of NaHCO₃ was added and the aqueous phase was extracted 3 times with AcOEt. The combined organic phases were washed with saturated NaCl solution, dried over MgSO₄, filtered and concentrated in vacuo. The residue was eluted on a silica gel column with 0 to 40% AcOEt in hexane to obtain (16) (440 mg, 87%) as a white solid. ¹H NMR (DMSO-d₆, 400 MHz): □ 9.58; (s, 1H, OH), 8.61; (s, 1H, H-ar), 8.17; (s, 1H, H-ar), 8.14; (d, 1H, J=8.6 Hz, H-ar), 8.06; (d, 1H, J=8.7 Hz, H-ar), 7.97; (dd, 1H, J=1.1 Hz, J=8.6 Hz, H-ar), 7.86; (dd, 1H, J=0.9 Hz, J=8.6 Hz, H-ar), 7.52-7.48; (m, 2H, 2 H-ar), 6.92; (d, 1H, J=8.2 Hz, H-ar), 3.91; (s, 3H, OCH₃), 2.17; (s, 6H), 2.06; (s, 3H), 1.75; (s, 6H). ¹³C NMR (DMSO-d₆, 100.6 MHz) □ 166.4, 156.5, 140.9, 136.1, 135.6, 130.7, 130.2, 129.9, 129.8, 128.5, 126.0, 125.4, 125.2, 125.0, 123.6, 117.0, 52.1, 36.6, 36.4, 28.4.

### Methyl 6-(3-(adamantan-1-yl)-4-(prop-2-yn-1-yloxy)phenyl)-2-naphthoate (17)

To a solution of naphthoate (16) (100 mg, 0.24 mmol, 1 eq.) in anhydrous THF (2.4 ml, 0.1 M) under an argon atmosphere at 0° C. in a flask dried by flame was added NaH (60%, 12 mg, 0.29 mmol, 1.2 eq.). The obtained mixture was stirred for 5 min at 0° C. then propargyl bromide (33 μl, 0.29 mmol, 1.2 eq.) was then added. The solution, after reaching room temperature, was mixed for 3 hours. H₂O was added and the aqueous phase was extracted 3 times with CH₂Cl₂ and the combined organic phases were washed with saturated NaCl solution, dried over MgSO₄, filtered and concentrated in vacuo. The residue was eluted on a silica gel column with 5:95 AcOEt:Cyclohexane to obtain (17) (102 mg, 94%) as a white solid. ¹H NMR (CDCl₃, 400 MHz): □ 8.62; (s, 1H, H-ar), 8.07; (dd, 1H, J=1.6 Hz, J=8.6 Hz, H-ar), 8.01; (s, 1H, H-ar), 7.99; (d, 1H, J=8.6 Hz, H-ar), 7.92; (d, 1H, J=8.6 Hz, H-ar), 7.79; (dd, 1H, J=1.7 Hz, J=8.5 Hz, H-ar), 7.62; (d, 1H, J=2.3 Hz, H-ar), 7.54; (dd, 1H, J=2.3 Hz, J=8.4 Hz, H-ar), 2.55; (t, 1H, J=2.3 Hz, CHCCH₂), 2.20; (s, 6H), 2.12; (s, 3H), 1.81; (s, 6H). ¹³C NMR (CDCl₃, 100.6 MHz) □ 167.4, 157.0, 141.4, 139.6, 136.1, 133.7, 131.5, 131.0, 129.9, 128.4, 127.2, 126.6, 126.4, 125.8, 125.7, 125.0, 113.5, 78.9, 75.5, 55.9, 52.3, 40.9, 37.4, 37.2, 29.2.

### 6-(3-(adamantan-1-yl)-4-(prop-2-yn-1-yloxy)phenyl)-2-naphthoic acid (H EMO-035)

To a solution of (17) (100 mg, 0.22 mmol) in 2:1 THF:H2O (3 mL) at room temperature was added LiOH (13 mg, 0.55 mmol, 2.5 eq.). After mixing for 16 h, 1M HCl was added to reach pH=1. The formed precipitate was filtered, washed with H₂O and dried in vacuo to obtain (HEMO-035) (36 mg, 38%) as a white solid. ¹H NMR (DMSO-d₆, 500 MHz): □ 13.03; (s, 1H, OH), 8.62; (s, 1H, H-ar), 8.23; (s, 1H, H-ar), 8.16; (d, 1H, J=8.6 Hz, H-ar), 8.08; (s, 1H, H-ar), 7.98; (d, 1H, J=8.6 Hz, H-ar), 7.90; (d, 1H, J=8.6 Hz, H-ar), 7.66; (dd, 1H, J=1.3 Hz, J=8.4 Hz, H-ar), 7.60; (s, 1H, H-ar), 7.18 (d, 1H, 8.5 Hz, H-ar), 4.91 (d, J =1.6 Hz, H-ar), 3.60 (t, 1H, J =1.6 Hz, CHCCH₂), 2.16; (s, 6H), 2.08; (s, 3H), 1.77; (s, 6H). ¹³C NMR (DMSO-d₆, 125.7 MHz) □ 167.5, 156.5, 140.1, 138.5, 135.5, 132.3, 131.0, 130.3, 129.8, 128.4, 127.7, 126.0, 125.6, 125.5, 125.3, 124.3, 114.0, 79.3, 78.1, 67.0, 56.6, 40.1, 36.6, 36.5, 28.4. HRMS (ESI/Q-TDE) m/z calc. for C₃₀H₂₇O₃ [M−H]⁻ 435.1966, obtained 435.1962.

### Compound H38

The compound H38 (HEMO-038) of formula:

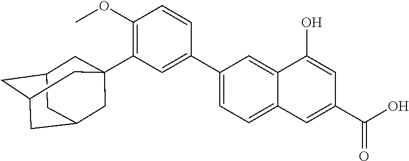 [SMILES: COc1ccc(-c2ccc3cc(C(=O)O)cc(O)c3c2)cc1C12CC3CC(CC(C3)C1)C2]

is prepared according to the following reaction scheme:

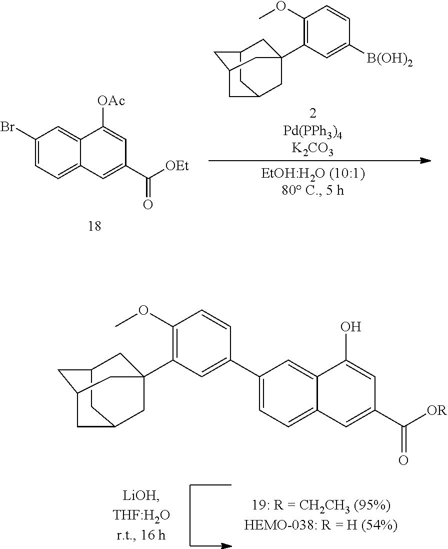

### Ethyl 6-(3-(adamantan-1-yl)-4-methoxyphenyl)-4-hydroxy-2-naphthoate (19)

Boronic acid (2) (150 mg, 0.53 mmol, 1 eq.) was added to a flame-dried reactor followed by bromoester (18) (179 mg, 0.53 mmol, 1 eq.), Pd(PPh₃)₄ (31 mg, 0.027 mmol, 5 mol %), and K₂CO₃ (146 mg, 1.06 mmol, 2 eq.). 10:1 EtOH:H₂O (4.4 mL, 120 mM) was added and the reactor was sealed. The resulting mixture was stirred at 80° C. for 5 hours. After reaching room temperature, H₂O and CH₂Cl₂ were added. The aqueous phase was extracted 3 times with CH₂Cl₂, and the combined organic phases were washed with saturated NaCl solution, dried over MgSO₄, filtered and concentrated in vacuo. The residue was triturated in cold CH₂Cl₂ and the resulting precipitate was filtered and dried in vacuo to obtain (19) (142 mg, 59%) as a white solid. ¹H NMR (DMSO-d₆, 500 MHz): □ 10.55; (s, 1H, OH), 8.31; (s, 1H, H-ar), 8.08; (s, 1H, H-ar), 8.06; (d, 2H, J=8.4 Hz, H-ar), 7.85; (d, 1H, J=8.4 Hz, H-ar), 7.60; (d, 1H, J=8.1 Hz, H-ar), 7.53; (s, 1H, H-ar), 7.41; (s, 1H, H-ar), 7.10; (d, 1H, J=8.4 Hz, H-ar), 4.35; (q, 2H, J=6.9 Hz, OCH₂), 3.85; (s, 3H, OCH₃), 2.12; (bs, 6H), 2.05; (bs, 3H), 1.74; (bs, 6H), 4.35; (t, 2H, J=7.1 Hz, CH₃). ¹³C NMR (DMSO-d₆, 125.7 MHz) □ 166.0, 158.5, 153.5, 139.3, 138.0, 132.2, 131.9, 129.8, 127.2, 127.1, 126.2, 125.6, 124.9, 121.0, 118.3, 112.8, 106.9, 60.7, 55.3, 40.1, 36.6, 36.5, 28.4, 14.3.

### 6-(3-(adamantan-1-yl)-4-methoxyphenyl)-4-hydroxy-2-naphthoic acid (HEMO-038)

To a suspension of ester (19) (25 mg, 0.055 mmol) in 2:1 THF:H₂O (1.5 mL) was added LiOH (3 mg, 0.11 mmol, 2.5 eq.). After mixing for 4 h at 60° C., 1M HCl was added to reach pH=1. The obtained precipitate was filtered, washed with H₂O and dried in vacuo to obtain (HEMO-038) (12.3 mg, 52%) as a light yellow solid. ¹H NMR (DMSO-d₆, 400 MHz): □ 12.86; (bs, 1H, OH), 10.47; (s, 1H, OH), 8.29; (s, 1H, H-ar), 8.06; (s, 1H, H-ar), 8.05; (d, 2H, J=7.8 Hz, H-ar), 7.84; (d, 1H, J=8.7 Hz, H-ar), 7.61; (d, 1H, J=8.0 Hz, H-ar), 7.54; (s, 1H, H-ar), 7.38; (s, 1H, H-ar), 7.13; (d, 1H, J=8.6 Hz, H-ar), 3.87; (s, 3H, OCH3), 2.13; (bs, 6H), 2.07; (bs, 3H), 1.76; (bs, 6H). ¹³C NMR (DMSO-d₆, 100.6 MHz) □ 167.6, 158.5, 153.4, 139.0, 138.1, 132.2, 132.0, 129.7, 128.2, 127.0, 126.0, 125.6, 124.9, 121.1, 118.3, 112.8, 107.4, 53.4, 40.1, 36.6, 36.5, 28.4. HRMS (ESI/Q-TDE) m/z calc. for C281-12704 [M−H]⁻ 427.1915, obtained 427.1911.

### Compound H39

The compound H39 (HEMO-039) of formula:

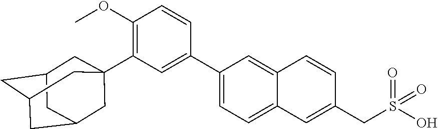 [SMILES: COc1ccc(-c2ccc3cc(CS(=O)(=O)O)ccc3c2)cc1C12CC3CC(CC(C3)C1)C2]

is prepared according to the following reaction scheme:

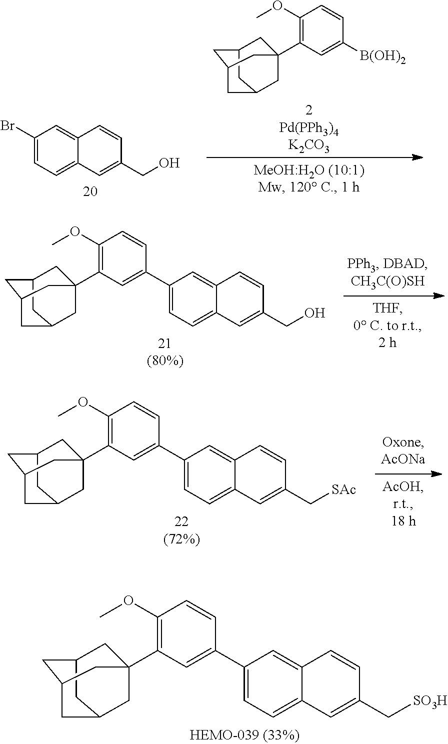

### (6-(3-(adamantan-1-yl)-4-methoxyphenyl)naphthalen-2-yl)methanol (21)

Boronic acid (2) (150 mg, 0.53 mmol, 1 eq.) was added to a flame-dried microwave reactor followed by bromonaphthol (20) (126 mg, 0.53 mmol, 1 eq.), Pd(PPh₃)₄ (31 mg, 0.027 mmol, 5 mol %), and K₂CO₃ (146 mg, 1.06 mmol, 2 eq.). 10:1 MeOH:H₂O (4.4 mL, 120 mM) was added and the reactor was sealed. The mixture obtained was stirred at 120° C. for 1 hour in the microwave. After reaching room temperature, H₂O and CH₂Cl₂ were added. The aqueous phase was extracted 3 times with CH₂Cl₂, and the combined organic phases were washed with saturated NaCl solution, dried over MgSO₄, filtered and concentrated in vacuo. The residue was eluted on a silica gel column with 100% CH₂Cl₂ to obtain (21) (167 mg, 80%) as a white solid. ¹H NMR (CDCl₃, 400 MHz): □ 7.98; (s, 1H, H-ar), 7.90-7.82; (m, 4H, 4 H-ar), 7.74; (d, 1H, J=8.5 Hz, H-ar), 7.60; (d, 1H, J=1.7 Hz, H-ar), 7.55-7.48; (m, 2H, 2 H-ar), 6.99; (d, 1H, J=8.4 Hz, H-ar), 4.87; (s, 2H, OCH₂), 3.90; (s, 3H, OCH₃), 2.20; (bs, 6H), 2.11; (bs, 3H), 1.81; (bs, 6H). ¹³C NMR (CDCl₃, 100.6 MHz) □ 158.7, 139.2, 139.0, 138.1, 133.4, 133.2, 132.3, 128.6, 128.4, 126.2, 126.0, 125.7, 125.6, 125.4, 125.0, 112.2, 65.7, 55.3, 40.8, 37.3, 29.3. HRMS (ESI/Q-TDE) m/z calc. for C₂₈H₃₀O₂ K [M+K]⁺ 437.1877, obtained 437.1869.

### (6-(3-(adamantan-1-yl)-4-methoxyphenyl)naphthalen-2y1)methyl)ethanethioate (22)

To a solution of PPh₃ (68 mg, 0.26 mmol, 2 eq.) in THF (500 μl) at 0° C. under argon atmosphere was added di-tert-butyl azodicarboxylate (60 mg, 0.26 mmol, 2 eq.) and the resulting solution was stirred for 30 min at 0° C. A solution of alcohol (21) (50 mg, 0.13 mmol, 1 eq.) in THF (500 μl) was then added and the mixture obtained was stirred for 1 hour at 0° C. then for 1 hour at room temperature. After dilution with CH₂Cl₂, the organic phase was washed with 1M HCl. The aqueous phase was extracted 3 times with CH₂Cl₂ and the combined organic phases were dried over MgSO₄, filtered and concentrated in vacuo. The residue was eluted on a silica gel column with 3:1 CH₂Cl₂:Cyclohexane to obtain (22) (43 mg, 72%) as a white solid. ¹H NMR (CDCl₃, 400 MHz): □ 7.95; (d, 1H, J=0.6 Hz, H-ar), 7.83; (d, 1H, J=8.4 Hz, H-ar), 7.76; (s, 1H, H-ar), 7.72; (dd, 1H, J=1.6 Hz, J=8.5 Hz, H-ar), 7.58; (d, 1H, J=2.3 Hz, H-ar), 7.52; (dd, 1H, J=2.3 Hz, J=8.4 Hz, H-ar), 7.39; (dd, 1H, J=1.6 Hz, J=8.4 Hz, H-ar), 6.99; (d, 1H, J=8.4 Hz, H-ar), 4.30; (s, 2H, SCH₂), 3.90; (s, 3H, OCH₃), 2.38; (s, 3H, SAc), 2.19; (bs, 6H), 2.10; (bs, 3H), 1.81; (bs, 6H). ¹³C NMR (CDCl₃, 100.6 MHz) □ 195.3, 158.8, 139.2, 139.0, 134.7, 133.2, 133.1, 132.3, 128.8, 128.2, 127.4, 127.3, 126.2, 126.0, 125.7, 124.9, 112.3, 55.3, 40.8, 37.3, 34.0, 30.5, 29.3.

### (6-(3-(adamantan-1-yl)-4-methoxyphenyl)naphthalen-2-yl)methanesulfonic acid (HEMO-039)

Thioacetate (22) (43 mg, 0.094 mmol, 1 eq.) was suspended in AcOH (1.07 mL, 87.5 mM). AcONa (77 mg, 0.94 mmol, 10 eq.) was added, followed by potassium hydrogen persulfate (74 mg, 0.24 mmol, 2.5 eq.) and the mixture obtained was stirred vigorously for 16 h. AcOEt was added and the precipitate obtained was filtered and washed with AcOEt then the organic phases were concentrated in vacuo. The residue was eluted on a silica gel column with 0 to 20% MeOH in CH₂Cl₂ to obtain (HEMO-039) (14.5 mg, 33%) as a white solid. ¹H NMR (DMSO-d₆, 400 MHz): □ 8.08; (s, 1H, H-ar), 7.89; (d, 1H, J=8.7 Hz, H-ar), 7.86; (d, 1H, J=8.4 Hz, H-ar), 7.77-7.74; (m, 2H, 2 H-ar), 7.62; (dd, 1H, J=2.2 Hz, J=8.4 Hz, H-ar), 7.56; (d, 1H, J=2.3 Hz, H-ar), 7.52; (dd, 1H, J=1.5 Hz, J=8.4 Hz, H-ar), 7.10; (d, 1H, J=8.6 Hz, H-ar), 3.88; (s, 2H, SCH₂), 3.86; (s, 3H, OCH₃), 2.14; (bs, 6H), 2.07; (bs, 3H), 1.76; (bs, 6H). ¹³C NMR (DMSO-d₆, 100.6 MHz) □ 158.2, 137.9, 137.1, 133.1, 132.3, 132.1, 131.7, 129.4, 128.1, 128.0, 126.9, 125.4, 124.8, 123.9, 112.7, 57.7, 55.3, 36.6, 28.4. HRMS (ESI/Q-TDE) m/z calc. for C₂₈H₂₉O₄S [M−H]⁻ 461.1792, obtained 461.1784.

### Example 4—Synthesis of Compound H24

The compound H24 of formula:

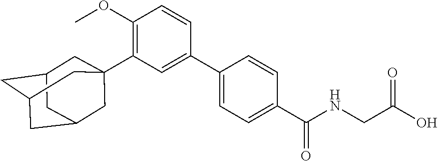 [SMILES: COc1ccc(-c2ccc(C(=O)NCC(=O)O)cc2)cc1C12CC3CC(CC(C3)C1)C2]

is prepared according to the following reaction scheme:

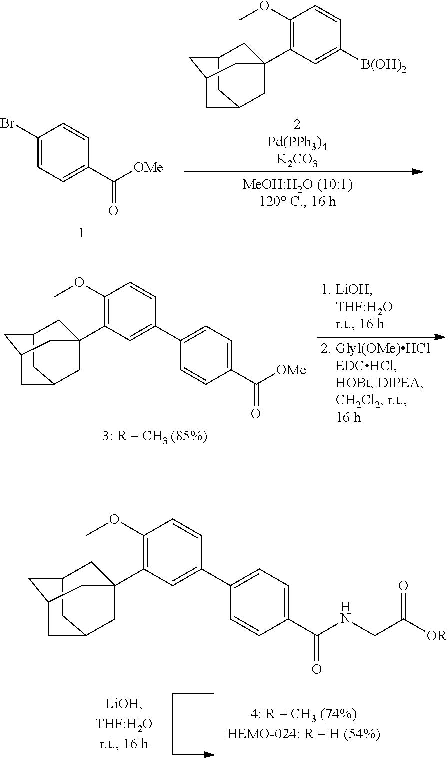

### Methyl 3′-(adamantan-1-yl)-4′-methoxy[1,1′-biphenyl]-4-carboxylate (3)

Boronic acid (2) (242 mg, 0.85 mmol, 1 eq.) was added to a flame-dried reactor followed by methyl 4-bromobenzoate (1) (183 mg, 0.85 mmol, 1 eq.), Pd(PPh₃)₄ (54 mg, 0.047 mmol, 5 mol %), and K₂CO₃ (257 mg, 1.86 mmol, 2 eq.). After addition of 10:1 MeOH:H₂O (7.75 mL, 120 mM) the reactor was sealed. The solution was then stirred at 80° C. for 16 h. After reaching room temperature, H₂O and CH₂Cl₂ were added. The aqueous phase was extracted 3 times with CH₂Cl₂, and the combined organic phases were washed with saturated NaCl solution, evaporated over MgSO₄, filtered and concentrated in vacuo. The residue was eluted on a silica gel column with 1:2 CH₂Cl₂:Cyclohexane to give (3) (260 mg, 85%) as a white solid. ¹H NMR (CDCl₃, 400 MHz): □ 8.07; (d, 2H, J=8.4 Hz, 2 H-ar), 7.63; (d, 2H, J=8.4 Hz, 2 H-ar), 7.50; (d, 1H, J=2.3 Hz, H-ar), 7.45; (dd, 1H, J=2.3 Hz, J=8.4 Hz, 2 H-ar), 6.96; (d, 1H, J=8.4 Hz, H-ar), 3.93; (s, 3H, CH₃), 3.89; (s, 3H, CH₃), 2.15; (bs, 6H), 2.09; (bs, 3H), 1.79; (bs, 6H). ¹³C NMR (CDCl₃, 100.6 MHz) □ 167.3, 159.3, 146.2, 139.1, 132.1, 130.2, 128.1, 126.7, 125.9, 125.7, 112.2, 55.3, 52.2, 40.7, 37.2, 29.2, 27.1. HRMS (ESI/Q-TDE) m/z calc. for C₂₅H₂₉O₃ [M+H]⁺ 377.2111, found 377.2111.

### 2-(3′-(adamantan-1-yl)-4′-methoxy-[1,1′-biphenyl]-4-ylcarboxam ido) methyl acetate (4)

To a suspension of ester (3) (112 mg, 0.31 mmol) in 2:1 THF:H₂O (4.3 mL) was added LiOH (19 mg, 0.78 mmol, 2.5 eq.). After mixing for 18 h, 1M HCl was added to reach pH=1. The precipitate formed was filtered, washed with H₂O and evaporated in vacuo before being suspended in anhydrous CH₂Cl₂ (7.5 ml). EDC.HCl (140 mg, 0.73 mmol, 2.5 eq.), HOBt (120 mg, 0.87 mmol, 3 eq.) and DIPEA (200 μl, 1.15 mmol, 4 eq.) were then added and the mixture obtained was stirred for 5 min at room temperature before the addition of glycine methyl ester (74 mg, 0.58 mmol, 2 eq.). After stirring for 16 h, 1M HCl was added and the aqueous phase was extracted 3 times with CH₂Cl₂. The combined organic phases were washed with H₂O and saturated NaCl solution, evaporated over MgSO₄, filtered and concentrated in vacuo. The residue was eluted on a column of silica gel with 30 to 50% AcOEt in Cyclohexane to obtain (4) (97 mg, 74%) as a white solid. ¹H NMR (CDCl₃, 500 MHz): □ 7.87; (d, 2H, J=8.3 Hz, 2 H-ar), 7.64; (d, 2H, J=8.3 Hz, 2 H-ar), 7.48; (d, 1H, J=2.2 Hz, H-ar), 7.44; (dd, 1H, J=2.3 Hz, J=8.4 Hz, 2 H-ar), 6.96; (d, 1H, J=8.4 Hz, H-ar), 6.68; (bs, 1H, NH), 4.28; (d, 2H, J=4.9 Hz, NCH₂) 3.89; (s, 3H, CH₃), 3.82; (s, 3H, CH₃), 2.15; (bs, 6H), 2.09; (bs, 3H), 1.79; (bs, 6H). ¹³C NMR (CDCl₃, 125.7 MHz) □ 170.8, 167.5, 159.2, 145.2, 139.1, 132.0, 131.5, 127.7, 126.9, 125.8, 125.6, 112.2, 55.3, 52.6, 41.9, 40.7, 37.3, 37.2, 29.2. HRMS (ESI/Q-TDE) m/z calc. for C₂₇H₃₂NO₄ [M+H]⁺ 434.2326, obtained 434.2322.

### 2-(3′-(adamantan-1-yl)-4′-methoxy-[1,1′-biphenyl]-4-ylcarboxamido)acetic acid (HEMO-024, or H24)

To a suspension of ester (4) (25 mg, 0.058 mmol) in 2:1 THF:H₂O (1.5 mL) was added LiOH (4 mg, 0.15 mmol, 2.5 eq.). After stirring for 18 h, 1M HCl was added to reach pH=1. The precipitate formed was filtered, washed with H₂O and evaporated in vacuo to obtain (HEMO-024) (13 mg, 54%) as a white solid. ¹H NMR (DMSO-d₆, 400 MHz): □ 12.57; (bs, 1H, OH), 8.84; (t, 1H, J=5.9 Hz, NH), 7.93; (d, 2H, J=8.4 Hz, 2 H-ar), 7.72; (d, 2H, J=8.4 Hz, 2 H-ar), 7.55; (dd, 1H, J=1.9 Hz, J=8.4 Hz, H-ar), 7.46; (d, 1H, J=1.9 Hz, H-ar), 7.08; (d, 1H, J=8.6 Hz, H-ar), 3.94; (d, 2H, J=5.8 Hz, NCH₂), 3.85; (s, 3H, OCH₃), 2.11; (bs, 6H), 2.06; (bs, 3H), 1.75; (bs, 6H). ¹³C NMR (DMSO-d₆, 125.7 MHz) □ 171.4, 166.3, 158.7, 143.4, 138.0, 131.7, 131.1, 127.9, 126.0, 125.5, 124.8, 112.7, 55.4, 41.2, 36.6, 28.4. HRMS (ESI/Q-TDE) m/z calc. for C₂₆H₂₈NO_{4 [}M−H]⁻ 418.2024, obtained 418.2016.

### Example 5—Ex Vivo Thrombin Generation Assay

A second thrombin generation test is carried out, for the compounds H27, H31, H32, H35, H38, H39 and the compound H24.

The thrombin generation test was carried out at 37° C. using a CAT analyzer (Diagnostica Stago) with the PPP Reagent LOW reagent (Diagnostica Stago) in accordance with the manufacturer's instructions and according to the method established by Pr. Hemker in 2003. PPP Reagent LOW is a mixture of tissue factor (TF) 1 pM with phospholipid vesicles (PL, 4 μM). The test is triggered by the addition of a mixture of calcium+fluorescent substrate (ZGGR-AMC) (FluCa mixture).

From a technical point of view, 80 μL of surcharged plasma sample is added to 20 μL of «initiator complex» (PPP Reagent LOW) and incubated for 10 min at 37° C. Then, the reaction is triggered by adding 20 μL of FluCa (mixture of CaCl₂ and ZGGR-AMC substrate) and the fluorescence signal is measured over time. Each test is performed in triplicate.

For each tested molecule, 247 μL of plasma from the hemophiliac patient were surcharged with 13 μL of a solution containing different amounts of compound (molecule to be tested, factor VIII or dilution buffer) to obtain the desired final concentration (systematic dilution of the molecule to be tested in the plasma at 1/20). For example, to obtain the final concentration of 50 μM in plasma, 13 μL of a 1 mM solution was added to 247 μL of plasma. In parallel with the tests on the different chemical compounds, the plasma of a hemophiliac patient is surcharged with the same buffer for diluting the compound, an 8% solution of dimethyl sulfoxide (DMSO), as a negative control (0.4% of final DMSO) in order to obtain the basal level of thrombin generation of the used plasma. The positive control is the same plasma surcharged with plasma factor VIII (Factane, LFB, France) at a concentration of 1 IU/mL (or 100% factor VIII) in order to obtain the expected «normal» level of thrombin generation used plasma.

For this example, solutions of adapalene and 1 mM compounds were prepared with the buffer (18 mM HEPES, 135 mM NaCl, pH 7.35) instead of water as in Example 1. This modification has made it possible to increase the amount of thrombin generated by the plasma of the hemophiliac patient A in the presence of 50 μM of adapalene.

Compounds were tested at a final concentration of 50 μM in plasma, and signal was measured over time. The obtained results, expressed as the amount of thrombin measured at the maximum of the peak obtained and normalized to the thrombin peak obtained with adapalene, are shown in Table 1 below:

TABLE 1

| | | | | | | | | | Negative | Positive |
|---|---|---|---|---|---|---|---|---|---|---|
| Compound | Ad | H24 | H27 | H31 | H32 | H35 | H38 | H39 | control | control |
| Amount of | 1 | 0.23 | 0.51 | 0.62 | 0.55 | 0.53 | 0.96 | 1.22 | 0.13 ± | 0.28 ± |
| thrombin | | | | | | | | | 0.02 | 0.06 |

All the tested compounds according to the invention make it possible to obtain a significant increase in the generation of thrombin compared to the negative control, with, for all of them except the compound H24, a peak of generated thrombin higher than that of the control positive, and in particular much higher for the compounds Ad, H38 and H39.

1. A method of treating hemophilia comprising administering a compound of a general formula (III′) or a pharmaceutically acceptable salt thereof to a subject suffering from hemophilia: 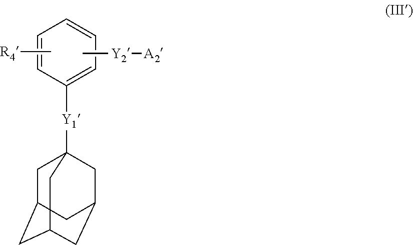
in which
Y₁′ represents a covalent bond or an amide group,
R₄′ represents a hydrogen atom, a hydroxyl group, a halogen atom, an amine group or a linear or branched, saturated or unsaturated carbon radical, which is optionally interrupted and/or substituted by one or more heteroatoms and/or one or more several groups including at least one heteroatom,
Y₂′ represents a covalent bond or an amide group,
A₂′ represents an optionally substituted cyclic or heterocyclic group including two fused rings, at least one of said rings being aromatic.

2. The method according to claim 1, for the restoration of coagulation in the plasma of the subject suffering from hemophilia.

3. The method according to claim 2, for the restoration of the generation of thrombin in the plasma of the subject suffering from hemophilia.

4. The method according to claim 1, according to which, in the general formula (III′), R₄′ represents an —OR₈ group or an —O—CO—R₈ group, where R₈ represents a linear or branched, saturated or unsaturated hydrocarbon radical, including from 1 to 10 carbon atoms, optionally substituted by one or two identical or different substituents R₁₄, R₁₄′, each selected from —F, —CO₂H, —SO₃H, —P(O)(OH)₂, —P(O)(OCH₃)₂, —P(O)(OCH₃)₂, —P(O)(OCH₂CH₃)₂, —N(CH₃)₂, —N(CH₂—CH₃)₂, 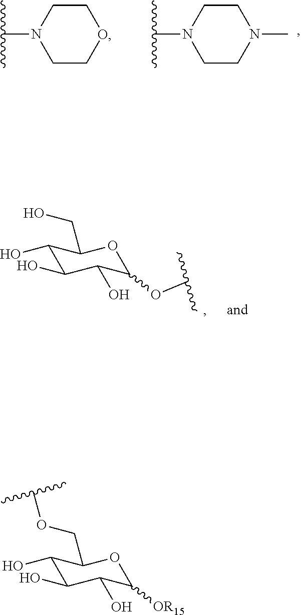
where R15 represents a hydrogen atom or a methyl group.

5. The method according to claim 4, according to which R₈ represents a group of general formula (XVIII): 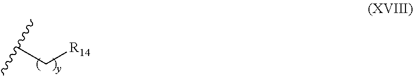
wherein y is an integer comprised between 1 and 10 and R₁₄ is as defined in claim 4.

6. The method according to claim 1, according to which, in the general formula (III′), R₄′ is fixed to the phenyl radical in the ortho or para position with respect to the adamantyl unit, and Y₂′ is fixed to the phenyl radical in the meta position relative to the adamantyl unit.

7. The method according to claim 1, according to which, in the general formula (III′), A₂′ is at least substituted by one substituent R₁₁ selected from fluorine, carboxyl, sulphonyl, phosphonyl, tetrazole or keto-oxadiazole, and linear, branched and/or cyclic, saturated or unsaturated, aromatic or non-aromatic carbon radicals, optionally interrupted and/or substituted by one or more heteroatoms, and/or one or more groups comprising at least a heteroatom.

8. The method according to claim 7, according to which R₁₁ is selected from the groups of formulas:

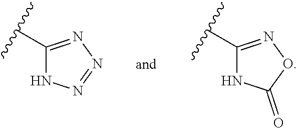 [SMILES: *c1nnn[nH]1 ; "and" ; *c1noc(=O)[nH]1]

9. The method according to claim 1, said compound corresponding to the general formula (IX): 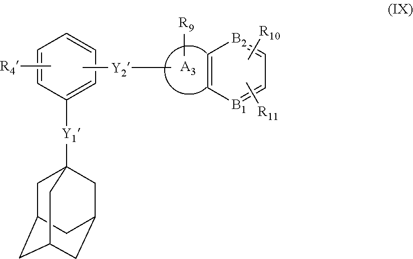
wherein
Y₁′, Y₂′ and R₄′ are as defined in claim 1,
A₃ represents a cyclic or 3- to 8-membered heterocyclic, saturated or unsaturated, aromatic or not hydrocarbon,
B₁ and B₂, which are identical or different, each represent a —CH— group or a nitrogen atom,
R₉ and R₁₀, which are identical or different, each represent a hydrogen atom, a hydroxyl group or an —OR₁₂ or —CO—O—R₁₂ group where R₁₂ represents a linear or branched, saturated or unsaturated hydrocarbon radical, including 1 to 10 carbon atoms, optionally substituted by one or two identical or different substituents R₁₆, R₁₆′, each selected from —F, —CO₂H, —SO₃H, —P(O)(OH)₂, —P(O)(OCH₃)₂, —P(O)(OCH₂CH₃)₂, —N(CH₃)₂, —N(CH₂—CH₃)₂, 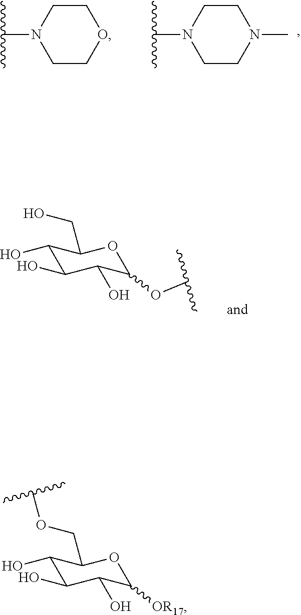
where R₁₇ represents a hydrogen atom or a methyl group,
and R₁₁ represents a substituent selected from fluorine, carboxyl, sulphonyl, phosphonyl, tetrazole or keto-oxadiazole groups, and linear, branched and/or cyclic, saturated or unsaturated, aromatic or not carbon radicals, which are optionally interrupted and/or substituted by one or more heteroatoms, and/or one or more groups including at least one heteroatom.

10. The method according to claim 9, wherein R11 represents a —(CH₂)ₓ—R₁₃ group where x is an integer comprised between 0 and 4 and R13 represents a fluorine atom or a carboxyl, sulfonyl, phosphonyl, tetrazole or keto-oxadiazole group.

11. The method according to claim 10, said compound corresponding to the general formula (X):

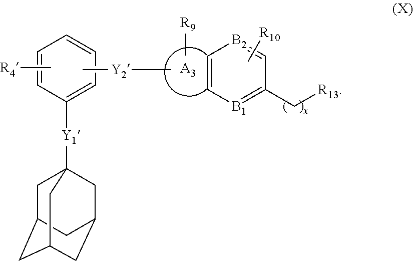

12. The method according to claim 10, said compound corresponding to the general formula (XI):

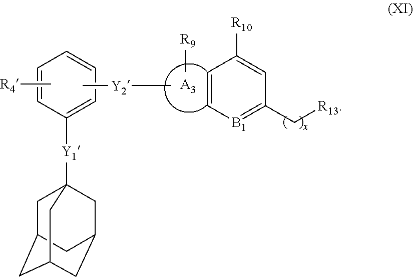

13. The method according to claim 10, said compound corresponding to the general formula (XII):

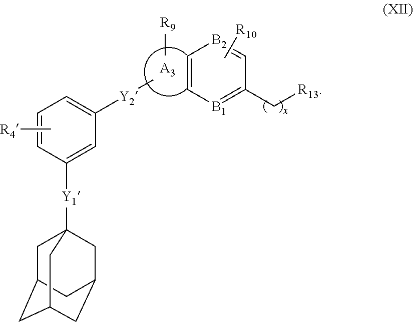

14. The method according to claim 10, said compound corresponding to the general formula (XIII)

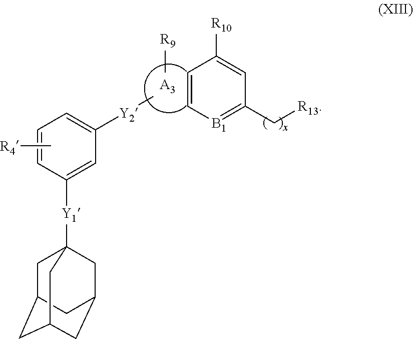

15. The method according to claim 10, said compound corresponding to the general formula (XIV):

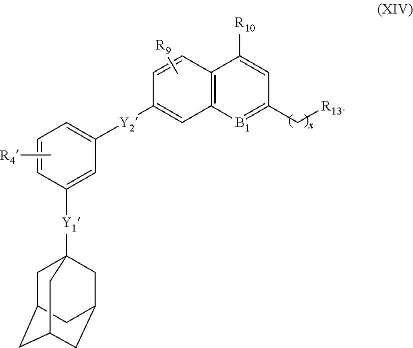

16. The method according to claim 10, said compound corresponding to the general formula (XV):

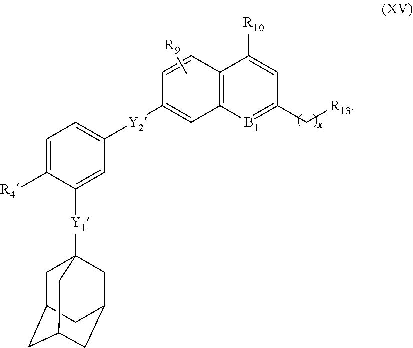

17. The method according to claim 10, said compound corresponding to the formula (XVI):

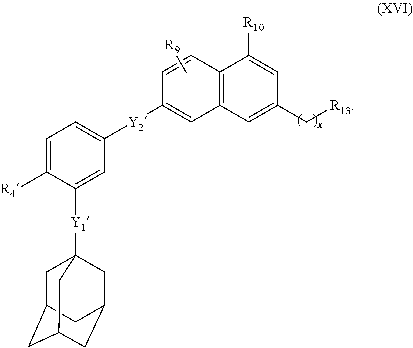

18. The method according to claim 1, said compound corresponding to the formula (V):

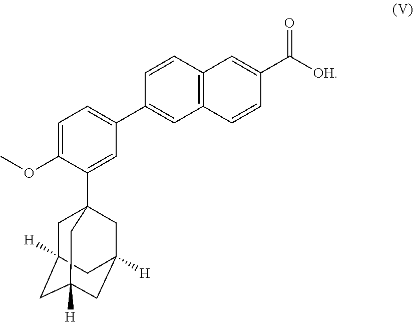 [SMILES: COc1ccc(-c2ccc3cc(C(=O)O)ccc3c2)cc1C12C[C@H]3C[C@@H](C1)C[C@@H](C2)C3]

19. The method according to claim 1, said compound corresponding to the formula (XVII):

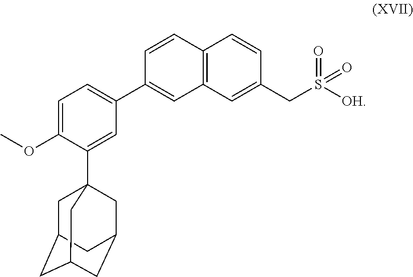 [SMILES: COc1ccc(-c2ccc3ccc(CS(=O)(=O)O)cc3c2)cc1C12CC3CC(CC(C3)C1)C2]

20. The method according to claim 1, wherein said subject is a mammal.

21. The method according to claim 1, wherein said compound is contained in a pharmaceutical composition, in a pharmaceutically acceptable vehicle.

22. The method according to claim 21, wherein said composition is in a form suitable for administration to said subject by oral route.


SEQUENCE LISTING (1 sequence)

SEQ ID NO: 1 - protein, 154 aa, Homo sapiens
DSEEDEEHTIITDTELPPLKLMHSGCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGF